(19) United States
(12) Patent Application Publication
(10) Pub. No.: US 2002/0123467 A1
(43) Pub. Date: Sep. 5, 2002

(54) Therapeutic composition comprising the KAL protein and use of the KAL protein for the treatment of retinal, renal, neuronal and neural injury

(75) Inventors: Christine Petit, Le Plessis-Robinson (FR); Nadia Soussi-Yanicostas, Paris (FR); Jean-Pierre Hardelin, Paris (FR); Catherine Sarailh, Marseille (FR); Genevieve Rougon, Marseille (FR); Renaud Legouis, Strasbourg (FR); Olivier Ardouin, Issy Les Moulineaux (FR); Jean-Claude Mazie, Asnieres (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: INSTITUT PASTEUR, Paris Cedex (FR)

(21) Appl. No.: 10/119,714

(22) Filed: Apr. 11, 2002

Related U.S. Application Data
(63) Continuation of application No. 09/319,236, filed on Sep. 2, 1999.
Application No. 09/319,236 is a 371 of international application No. PCT/EP97/06806, filed on Dec. 5, 1997.

(51) Int. Cl. A61K 38/17

(52) U.S. Cl. 514/12

(57) ABSTRACT

KAL protein is identified the active agent in a therapeutic composition for treatment of injury to nerve tissue including spinal cord tissue, as well as support of treatment for renal grafts. Additionally, therapeutic treatment of renal injury, and kidney transplantation and renal surgery, is effected by administration of KAL protein. The therapeutic agent may be administered locally, or intravenously. Retinal disorders may be similarly treated.

### BACKGROUND OF THE INVENTION

1. Field of the Invention

This invention pertains to the use of the KAL protein in a therapeutic composition and to the treatment of patients suffering from neural, retinal and renal insult.

2. Background of the Invention

Kallmann's syndrome (KS) refers to the association of hypogonadism with anosmia (or hyposmia). Hypogonadism in KS is due to gonadotropin-releasing hormone (GnRH) deficiency (Naftolin et al., 1971; Sherins and Howards, 1986). Anosmia has been related to the absence or hypoplasia of the olfactory bulbs and olfactory tracts (De Morsier, 1954). In animals, the existence of interactions between olfactory and reproductive functions has long been reported (Whitten, 1956 Bruce, 1959; McClintock, 1971). More recently, developmental links between the olfactory system and the GnRH neuroendocrine system have also been identified. Embryo logical studies in several species including mouse (Schwanzel-Fukuda and Pfaff, 1989; Wray-et al., 1989), monkey (Ronnekleiv and Resko, 1990), chicken (Murakami et al., 1991; Norgren and Lehman, 1991 Nurakami and Akai, 1996), newt (Murakami et al., 1992) and man (Schwanzel-Fukuda et al., 1995), have led to the conclusion that GnRH synthesizing neurons migrate from the olfactory epithelium to the brain during embryonic life. GnRH cells migrate along an olfactory epithelium-forebrain axis of nerve fibers. In mammals, migrating GnRH cells are primarily found in close association with the vomeronasal and terminal nerves (Schwanzel-Fukuda et al, 1992), whereas in the chicken they appear to ascend along the olfactory nerves themselves (Murakami et al., 1991). Ultimately, the GnRH neurons reach the preoptic and hypothalamic areas where the neurosecretion takes place. From these observations, it was first hypothesized that the “double clinical defect” observed in KS affected patients (i.e. hypogonadism and anosmia) could be related to a unique defect in the development process of both olfactory and GnRH neurons.

The study of a human 19 week old male fetus carrying a large Xp deletion, including the KAL gene responsible for the X-linked form of the disease, has shown that neither the GnRH neurons, nor the axon terminals of the olfactory, terminalis and vomeronasal neurons were present in the brain. Although GnRH cells and olfactory axons had left the olfactory epithelium, they had accumulated in the upper nasal area, on the peripheral side of the dura layer (Schwanzel-Fukuda et al., 1989). This observation indicated that the embryonic defect responsible for the X-linked KS did not involve the initial differentiation step of olfactory and GnRH neurons within the olfactory placode, but rather the subsequent migration pathway of olfactory axons and GnRH cells to the brain. Furthermore, some patients have unilateral renal aplasia (Wegenke et al., 1975).

The human KAL gene has been isolated by positional cloning strategies (Franco et al., 1991; Legouis et al., 1991; Hardelin et al., 1992). The gene encodes a 680 amino acid putative protein (SwissProt P23352) including a signal peptide. The deduced amino acid sequence provides no evidence for either a hydrophobic transmembrane domain or glycosyl phosphatidyl inositol anchorage, suggesting that the protein is extracellular.

The interspecies conservation of the KAL gene sequence has been explored by Southern blot analysis with human KAL cDNA probes. Cross hybridization was observed in various mammals and in the chicken (Legouis et al., 1993). The KAL orthologue has been isolated in the chicken (Legouis et al., 1993; Rugarli et al., 1993). Sequence comparison with the human KAL cDNA demonstrated an overall identity of 72%, with 75% identity at the protein level.

The expression of the KAL gene during embryonic development has been studied in the chicken by in situ hybridization (Legouis et al., 1993; Legouis et al., 1994; Rugarli et al., 1993). From embryonic day 2 (ED2) to ED8, the KAL gene is expressed in various endodermal, mesodermal and ectodermal derivatives, whereas from ED8 onwards, the expression is almost entirely restricted to definite neuronal populations in the central nervous system including mitral cells in the olfactory bulbs, Purkinje cells in the cerebellum, striatal, retinal and tectal neurons, most of which still express the gene after hatching. According to such a spatio-temporal pattern of expression, it is proposed that the KAL gene is involved both in morphogenetic events and in neuronal late differentiation and/or survival.

### SUMMARY OF THE INVENTION

There is no adequate treatment presently available that leads to specific growth and guidance of neurons which have been injured or have degenerated.

Surprisingly, the inventors have discovered that the purified KAL protein possess different in vitro biological activities including neuron growth activity, and neurite fasciculation activity as well as adhesion properties to cerebellar neurons the latter being mediated, at least in part, via the fibronectin type III of the KAL protein.

In addition the KAL protein is an appropriate substrate for neuronal survival. Given these properties, the KAL protein its biologically active derivatives, its receptor(s) and its ligands are relevant to neuronal regeneration:

survival

adhesion

growth

fasciculation

Consequently, an object of the present invention concerns the therapeutic use of KAL protein or one of its biologically active derivatives, alone or in combination with other ligands, in disease of central or peripheral nervous system including:

1. Nerve injury of traumatic, infectious, metabolic or inherited origin.

2. Spinal injury of traumatic, infectious, metabolic or inherited origin.

3. Retinal disorder graft in context of traumatic, infectious, metabolic or inherited origin.

Renal treatment based on the role of the KAL protein in kidney morphogenesis:

4. Renal disease, hypoplasia or agenesis of traumatic, infectious, metabolic or inherited origin. 5. Kidney transplantation and renal surgery.

The diseases giving rise to these conditions are varied is and include, among others, amyotrophic lateral sclerosis, multiple sclerosis, Parkinson's, injuries of traumatic origin, neurotrophic ulcers, macular degeneration, diabetes, leprosy and renal failure.

One subject of the present invention is a therapeutic composition comprising a pharmaceutically active amount of a protein selected among the group consisting of:

the purified KAL protein;

a protein having at least 80% homology in aminoacid sequence with the KAL protein; or a protein having at least 80% homology in aminoacid sequence with a purified biologically active part of the KAL protein;

a protein which is specifically recognized by antibodies directed against the purified KAL protein.

By “biologically active part” of the KAL protein is intended a peptide having an aminoacid sequence which is contained in the entire aminoacid sequence of the KAL protein and which peptide exhibits at least one of the following in vitro activities

survival activity for cells, and specifically for neurons;

growth promoting activity for neurons;

induction of neurite fasciculation;

adhesion function.

A particular biologically active part of the KAL protein consists in one or several of the four fibronectin type III repeat of the KAL protein (FIG. 9) alone or in combination one with each other that are obtained by transfection of a procaryotic or an eukaryotic cell, specifically a CHO cell with the corresponding encoding DNA that has been inserted in a suitable expression vector.

The therapeutic composition according to the invention is able to induce the recovery of the functional activity of the neuron-associated cells.

Thus, this therapeutic composition according to the present invention comprises either the KAL protein or one of its “biologically active derivatives” that are above defined.

Another subject of the present invention is a therapeutic composition containing a pharmaceutically effective amount of a polynucleotide sequence (RNA, genomic DNA or cDNA) coding for the purified KAL protein or a biologically active derivative of the KAL protein.

Another subject of the present invention is a method for cultivating neuronal cells in vitro comprising the addition of a biologically active amount of either the purified KAL protein, a protein having at least 80% homology in aminoacid sequence with the KAL protein or a purified biologically active part of the KAL protein to the cell culture medium.

Another subject of the present invention is a method for the production of the purified recombinant KAL protein comprising the steps of:

a) cultivating a prokaryotic or an eukaryotic cell that has been transfected with a vector carrying a DNA insert coding for the KAL protein, a purified biologically active part of the KAL protein or a protein which is recognized by antibodies directed against the purified KAL protein a purified biologically active part of the KAL protein or a protein which is recognized by antibodies directed against the purified KAL protein;

b) isolating the recombinant KAL protein from the culture preparation of the transfected prokaryotic and eukaryotic cell.

Another subject of the present invention is a method for screening ligands that binds to the KAL protein.

Another subject of the present invention is a method for screening molecules that modulates the expression of the KAL protein.

The KAL protein can be therapeutically administered in the form of a solution, gel or dry powder. It can be introduced locally. It can be administered intraveneously using devices that overcome the blood brain barrier.

### BRIEF DESCRIPTION OF THE DRAWINGS

FIG. 1: The KAL protein promotes the adhesion of cerebellar granule cells. Cerebellar granule cells were isolated from postnatal day-5 mice and were plated on plastic surfaces which were coated with KAL protein, or with BSA, or with laminin for 90 min at 37° C. as described in Materials and Methods. The wells were washed three times with PBS and the adherent cells were counted as described in Materials and Methods. Similar results were obtained in three separate experiments.

The results are expressed as the percentage of adherent cells, relative to the total number of cells deposited in the well.

FIG. 2: The KAL protein promotes the adhesion of PC12 cells. PC12 cells were plated on plastic surfaces which were coated with KAL protein, or with BSA, or with laminin for 90 min at 37° C. as described in materials and methods. The wells were washed three times with PBS and adherent cells were counted as described in Materials and Methods.

The results are expressed as the percentage of adherent cells, in relation to the total number of cells deposited on the substratum.

FIG. 3: Antibody-mediated inhibition of PC12 cell adhesion to the KAL protein. PC12 cells were plated on wells which had been previously coated with KAL protein and incubated in the presence of increasing concentrations of antiserum directed against the KAL protein. The number of adherent cells was calculated as described above. The results of three independent experiments are expressed as the percentage of adherent cells in presence of immune or preimmune sera, relative to the total number of cells deposited in the wells.

FIG. 4: Adhesion of PC12 cells to the KAL protein was inhibited in the presence of heparin. PC12 cells were added to the wells which had been previously coated with KAL protein and incubated in the presence of increasing concentrations of heparin, and then the number of adherent cells was calculated as described above. The results are expressed as the percentage of adherent cells in absence or in presence of heparin, in relation to the total number of cells deposited on the substratum.

FIG. 5: Adhesion of PC 12 cells to KAL protein was inhibited in the presence of R1.FNIII. PC12 cells were incubated with increasing concentrations of R1-FNIII, or human serum albumin (HSA) and added to the wells which have previously been coated with KAL protein. The number of adherent cells was calculated as described above. The results are expressed as the percentage of adherent cells in absence or in presence of R1-FNIII, relative to the total number of cells deposited on the substratum.

FIG. 6: Reaggregates of cerebellar neurons from postnatal day-5 are cultured for 48 h on KAL protein substrate (A), or respectively on positive or negative controls, poly-1-lysine (B), BSA (C). Cells were stained with toluidine blue. Note that KAL protein is a permissive substrate for survival and neurite outgrowth of cerebellar granule cells.

FIG. 7: Immunodetection of the KAL protein expressed in CHO cells. Wild type CHO cells (A), KAL-transfected clone 1-1 (B), and 2-3 (C). Cells were fixed using paraformaldehyde. Note that the immunostaining delineates the cells and displays the expected pattern for an extracellular matrix component.

FIG. 8: Induction of neurite fasciculation from cerebellar cell aggregates by a monolayer of KAL-expressing cells. Aggregates of cerebellar neurons from post-natal day 5 mice were cultured for 24 h on monolayers of either wild type CHO cells (A), or clones of KAL-transfected CHO cells, clone 2-3 (B-D) and clone 1-1 (E and F). Neurites were short and fasciculated on KAL-expressing cells (B, D and F). C and E: in the presence of anti-KAL Fab fragments (0.2 mg/ml) neurite fasciculation was not induced from cell aggregates cultured on KAL-transfected cells. Neurons were stained for GAP 43 immunoreactivity.

FIG. 9: Aggregates of cerebellar neurons cultured for 24 h on either wild type CHO cells (A) or clones of KAL-transfected CHO cells: clone 2-3 (B and C). Neurite fasciculation observed on KAL-expressing cells (B) is prevented by the addition of anti-KAL Fab to culture medium (C).

FIG. 10: Aminoacid sequence of the human KAL protein, the fibronectin type III repeats are respectively located in the following sequences:

the sequence beginning at aminoacid in position 182 and ending at aminoacid in position 286 of the entire aminoacid sequence of the human KAL protein;

the sequence beginning at aminoacid in position 287 and ending at aminoacid in position 403 of the entire aminoacid sequence of the human KAL protein;

the sequence beginning at aminoacid in position 404 and ending at aminoacid in position 541 of the entire aminoacid sequence of the human KAL protein;

the sequence beginning at aminoacid in position 542 and ending at aminoacid in position 662 of the entire aminoacid sequence of the human KAL protein.

FIG. 11: Schematic representation of the localization of the different domains of the KAL protein.

FIG. 12 to FIG. 17: The KAL promotes the adhesion of a variety of cell types.

Cells were plated on wells coated with purified KAL (3 μg/ml), or laminin (20 μg/ml), or BSA (10 mg/ml) for 60 min at 37° C. as described in Material and Methods.

Neuronal Cells:

FIG. 12: rat olfactory neurons cell line, line 24;

FIG. 13: Mouse GnRH neurons cell line, line GT1;

FIG. 14: PS cerebellar granule cells;

FIG. 15: rat pheochromocytoma PC12 cells;

Non-Neuronal Cells:

FIG. 16: kidney epithelial cell line, line LLCPK;

FIG. 17: Chinese Hamster Ovary cell line.

FIG. 18: Inhibition of the adhesion of PC12 cells to KAL. PC12 cells were plated on KAL coated wells that were previously incubated with increasing concentrations of either preimmune serum (PIS) or immune serum (IS) directed against purified KAL.

The results are expressed as the percentage of cells adherent on the tested substrate, relative to the number of cells that adhere on a Poly-lysine substrate. The results are the mean of three independent experiments.

FIG. 19 to FIG. 23 Adhesion of CHO cells to KAL.

FIG. 19 to FIG. 20: Adhesion of CHO cells to KAL is inhibited by exogenous HS or CS.

Adhesion of CHO-K1 cells on KAL. CHO-K1 cells were plated on surfaces which were coated with purified KAL (3 mg/ml), or laminin (20 mg/ml), or BSA (10 mg/ml) for 60 min at 37° C. as described in Material and Methods.

FIG. 19: Adhesion of CHO-K1 cells on KAL in the presence of various concentrations of heparin.

FIG. 20: Adhesion of CHO-K1 cells on KAL in the presence of various concentrations of chondroitin sulfate.

FIG. 21 and FIG. 22: Adhesion of CHO cells to KAL is mediated by both HSPG and CSPG.

HSPG are required for efficient adhesion of CHO cells to KAL.

Wild-type CHO cells (CHO-K1), mutant CHO cells that expresses undersulfated cell surface HSPGs (CHO-606) or mutant CHO cells that lack cell surface HSPGs but overexpress chondroitin sulfate (CHO-677), were plated on surfaces which were coated with purified KAL (3 mg/ml), or with fibronectin (10 mg/ml), for 60 min at 37° C. as described in Material and Methods.

Chondroitinase treatment totally inhibits adhesion of HS-deficient cells adhesion on KAL.Wild-type CHO (CHO-K1) or HS-deficient cells (CHO-677) were incubated with increasing concentrations of chondroitinase ABC enzyme (only one concentration was shown) for 15 min at 37° C, prior plating cells on KAL substrate.

HS- and CS-deficient cells were unable to adhere to KAL. Wild-type CHO cells (CHO-K1), mutant HS- and CS-deficient cells (CHO-745), were plated on surfaces which were coated with purified KAL (3 mg/ml), or with fibronectin (10 mg/ml), for 60 min at 37° C. as described in Material and Methods.

The percentage of adherent cells are calculated as in FIG. 12.

FIG. 21: Adhesion of different CHO cell lines onto KAL or fibronectin.

FIG. 22: Adhesion of different CHO cell lines onto KAL in the presence of various concentrations of chondroitinase.

FIG. 23: Adhesion of different CHO cell lines onto KAL or fibronectin.

FIG. 24: Conservation of the first fibronectin type III repeat of KAL throughout evolution.

(A) Schematic representation of the structure of human KAL (del Castillo et al., 1992). The position of four fibronectine type III repeats (FNIII) are indicated. A black box indicated a “four disulphide core” domain. Also depicted the location of the 32 amino acids peptide (32R1) used in adhesion assays.

(B) Alignment of the sequence corresponding to peptide 32R1 sequence KAL in human (del Castillo et al., 1992), chicken (Legouis et al., 1993; Rugarli et al., 1993), quail (Legouis et al., 1993) and zebrafish (Ardouin et al unpublished). The identical amino acids are boxed.

FIG. 25 and 26: 32 R1 peptide contains a major cell binding site of KAL

FIG. 25: Adhesion of CHO-K1 to 32R1 substrate. CHO-K1 were plated on substra coated with KAL (3 mgml), peptide 32R1 (10 mgml), peptide C17 (10 mgml) or peptide C16V (10 mgml), as described in Material and Methods.

FIG. 26: Inhibition of cell adhesion to KALby 32 R1

CHO-K1 cells were incubated in the presence of KAL (0.35 μM), or 32 R1 (2.6 mM), or 17R2 (5mM) for 1 H at 4° C. prior plating on KAL coated wells.

The percentage of adherent cells was determined as in FIG. 1.

FIG. 27: Adhesion, spreading and neurite outgrowth of olfactory neurons (line 24) cultived on anosmin-1, peptide 32R1 or fibronectin substrates.

Olfactory neurons were maintained for one hour (A, B, C), eight hours (D, E, F) on anosmin-1 (A, D), or fibronectin (B, E) or peptide 32R1 (C, F) substrates. Adherent cells were fixed and stained with toluidine blue.

FIG. 28: Mean neurite length on the different substrates after one hour, two hours and eight hours in vitro. Mean value±SEM. More than 100 neurons were analyzed in each experimental condition.

### DETAILED DESCRIPTION OF THE INVENTION

The KAL protein has been produced in transfected eukaryotic cells, and specifically CHO cells. This protein with an approximate molecular mass of 100 kDa is N-glycosylated, secreted in the cell culture medium, and was found to be localized mainly at the cell surface. Therefore, the protein encoded by the KAL gene is likely to be an extracellular matrix component in vivo.

For the purpose of the present invention:

A “gene” refers to the entire DNA portion involved in the synthesis of a protein. A gene embodies the structural or coding portion which begins at the 5′ end from the translation start codon (usually ATG) and extends to the stop (TAG, TGA, or TAA) codon at the 3′ end. It also contains a promoter region, usually located 5′ or upstream to the structural gene, which initiates and regulates the expression of a structural gene. Also included in a gene are the 3′ end and poly(A)+addition sequences.

A “structural gene” is that portion of a gene comprising a DNA segment encoding a protein, polypeptide or a portion thereof, and excluding the 5′ and 3′ non coding sequences. Moreover, since heparin treatment of cell membrane fractions resulted in the release of the protein, we suggest that heparan-sulfate proteoglycans are involved in the binding of the protein to the cell membranes. Polyclonal and monoclonal antibodies: directed against the purified protein were generated. They subsequently allowed us to determine the cellular distribution of the protein in the chicken central nervous system at late stages of embryonic development. The protein is present on cell bodies and along neurites of definite neuronal populations including Purkinje cells in the cerebellum, mitral cells in the olfactory bulbs and several neuronal cell populations in the optic tectum and the striatum [Soussi-Yanicostas, 1996].

The N-terminal sequence of the KAL protein is cysteine-rich and can be subdivided into two subregions. The first has no similarity with any known protein. The other fits the consensus whey acidic protein (WAP) 4-disulfide core motif (Dandekar et al., 1982; Hennighausen and Sippel, 1982), a motif shared by several small proteins with serine protease inhibitory activity (Kato and Tominaga, 1979; Seemuller et al., 1986; Stetler et al., 1986; Wiedow et al., 1990). A particular feature of the C-terminus of the protein is the presence of 11 basic (including 6 histidyl) residues among 20 mostly hydrophilic amino acids. The KAL protein contains four contiguous fibronectin type III repeats (del Castillo et al., 1992). This motif has been found in a wide variety of molecules with morphoregulatory roles, most of which are involved in cell adhesion, fasciculation and growth of neurons. Among them, L1/NgCAM (Moos et al., 1988; Burgoon et al., 1991) Nr-CAM/Bravo (Grumet et al., 1991; Kayyem et al., 1992), F3/F11 (Gennarini et al., 1989; Brummendorf and Rathjen, 1993), TAG/Axonin-1 (Furley et al., 1990; Zuellig et al, 1992), Tenascin-R (Norenberg et al, 1995), Tenascin-C (Gotz et al., 1996). Interestingly, the type III repeats of the protein encoded by the KAL gene show even greater similarity with those of cell adhesion molecules such as TAG-1/Axonin-1, L1, and F3/F11 (Brummendorf and Rathjen, 1993) which have been shown to mediate neurite outgrowth or axon-axon interactions [Sonderegger and Rathjen, 1992 #48]. Altogether, the sequence comparisons suggest that the protein encoded by the KAL gene has several functions including protease inhibitory activity and adhesion.

We demonstrate that the purified KAL protein is a cell adhesion molecule that is permissive for neuron growth in vitro and is thus particularly suitable for neuron growth assays in vitro. We also show that transfected. CHO cells producing the KAL protein induce axonal fasciculation of cerebellar granule cells cultivated upon this CHO cell monolayer.

These results have allowed the inventors to design specific therapeutic compositions for treating various neuronal or renal disorders using the purified KAL protein or a biologically active derivative of the KAL protein as described above or, as an alternative embodiment, using a polynucleotide encoding for the KAL protein or for one of its biologically active derivative.

Among the purified biologically active parts of the KAL protein are proteins comprising at least one aminoacid sequence selected among the following sequences:

the sequence (SEQ ID n° 1) beginning at aminoacid in position 182 and ending at aminoacid in position 286 of the entire aminoacid sequence of the human KAL protein;

the sequence (SEQ ID n° 2) beginning at aminoacid in position 287 and ending at aminoacid in position 403 of the entire aminoacid sequence of the human KAL protein;

the sequence (SEQ ID n° 3) beginning at aminoacid in position 404 and ending at aminoacid in position 541 of the entire aminoacid sequence of the human KAL protein;

the sequence (SEQ ID n° 4) beginning at aminoacid in position 542 and ending at aminoacid in position 662 of the entire aminoacid sequence of the human KAL protein;

Furthermore, the inventors have also shown that another biologically active derivative of the KAL protein, namely a 32 aminoacids peptide (32R1) which is derived from the first fibronectin type III repeat of the KAL protein, inhibits adhesion of olfactory neurons line 24, PC12 cells and CHO cells to a KAL substrate.

The aminoacid sequence of the 32R1 peptide, which is also part of the present invention, is the following:

NH₂-RPSWYQFRVAAVNVHGTRGFTAPSKHFRSSK-COOH (SEQ ID n° 5).

This peptide may be therapeutically used, in general, as a biological glue.

In a preferred embodiment of the therapeutic compositions of the present invention, the amount of the biologically active peptide component is comprised in the range from 0.1 μg/ml to 10 4 μg/ml in the body fluid. The dose-range is expressed in reference to the bioavailability of the KAL protein or of one of its biologically active derivatives at the body site to be treated.

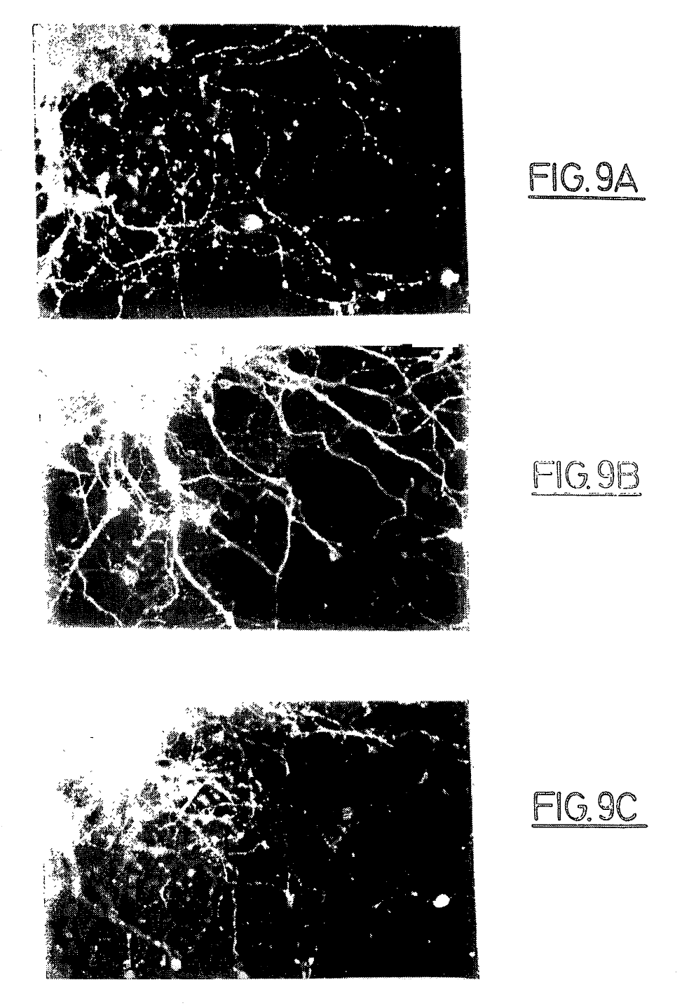

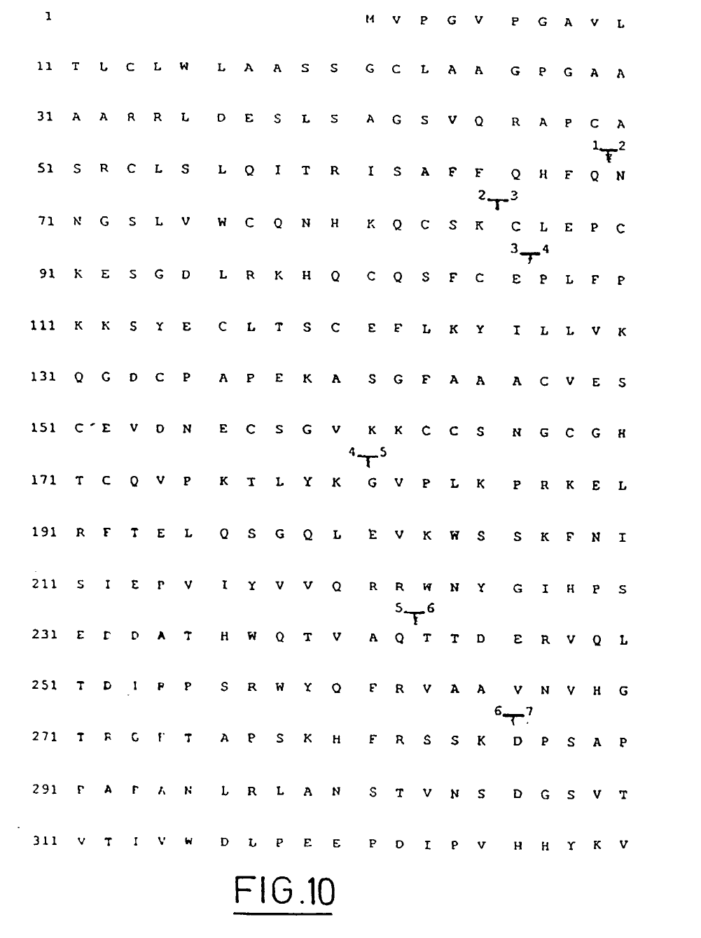

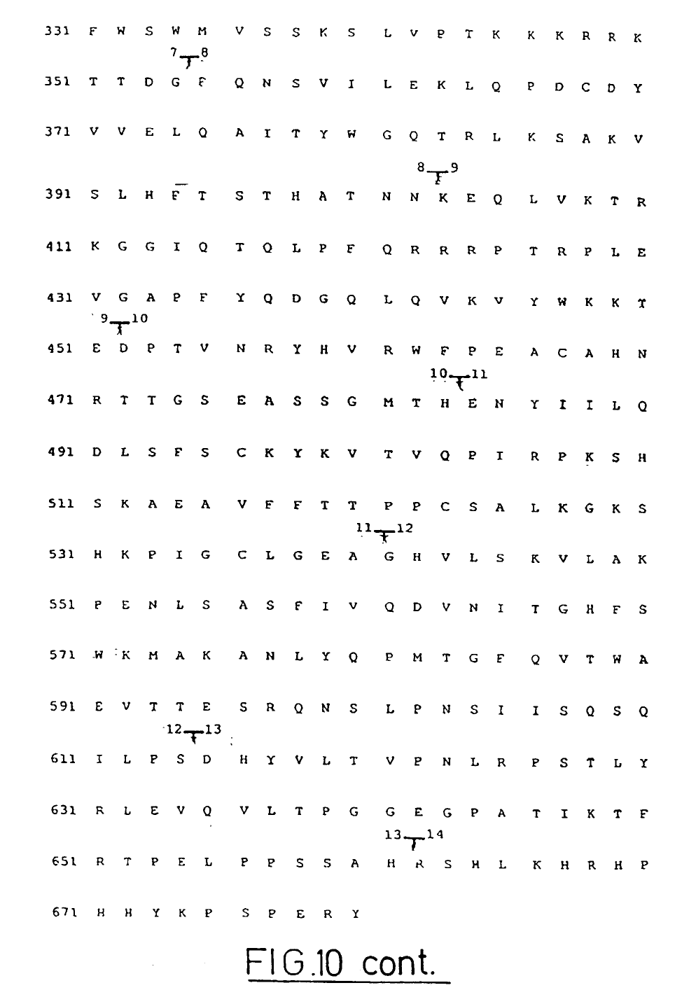

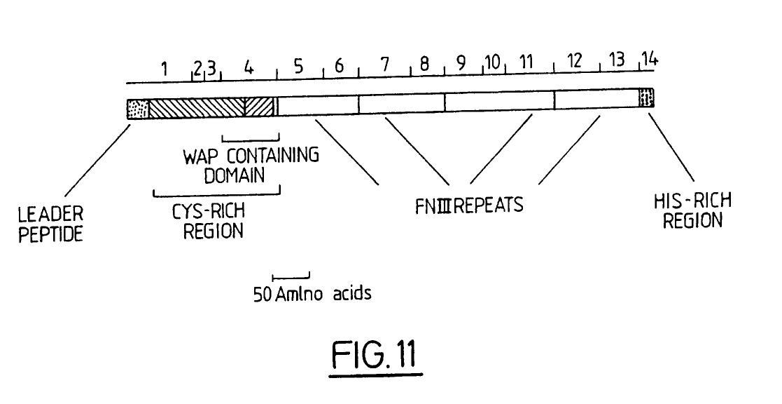

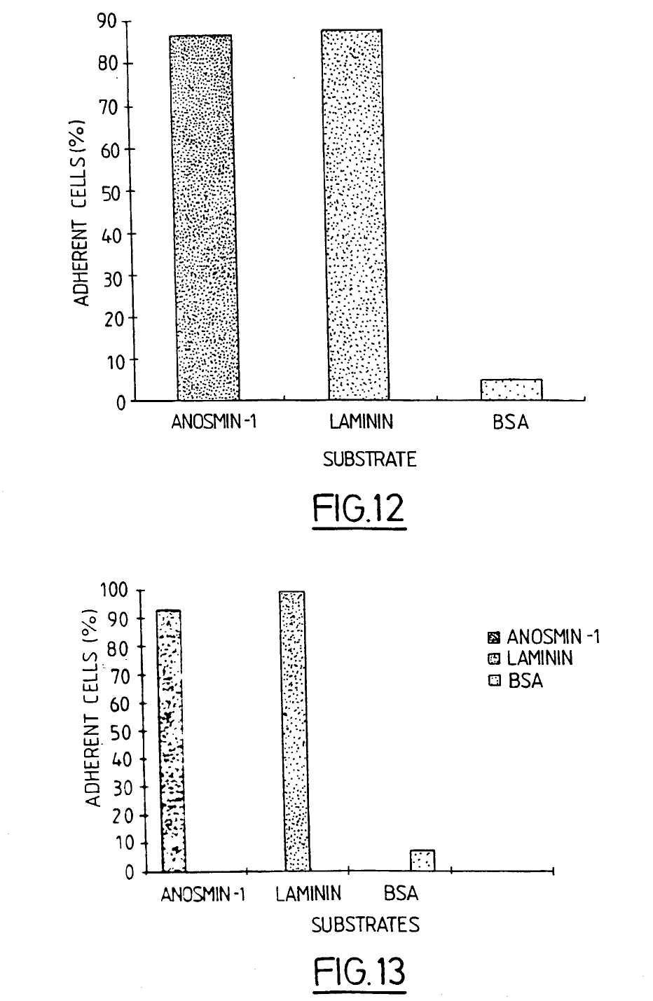

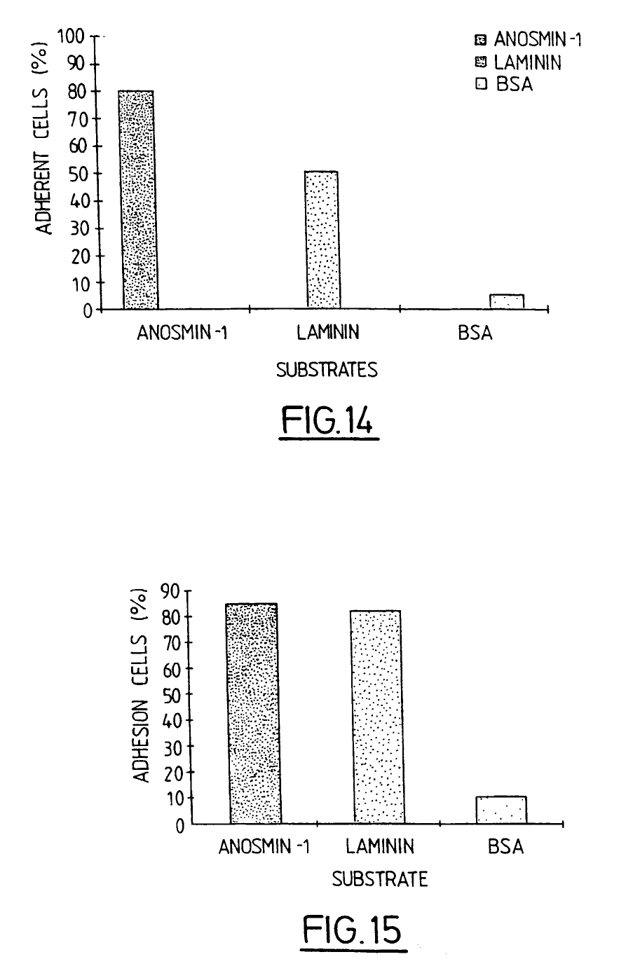

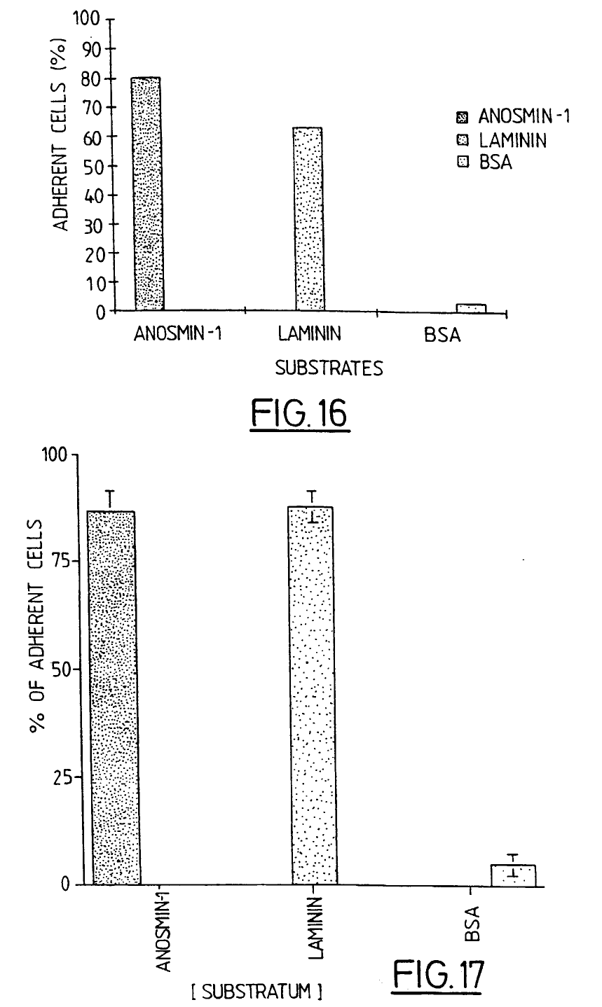

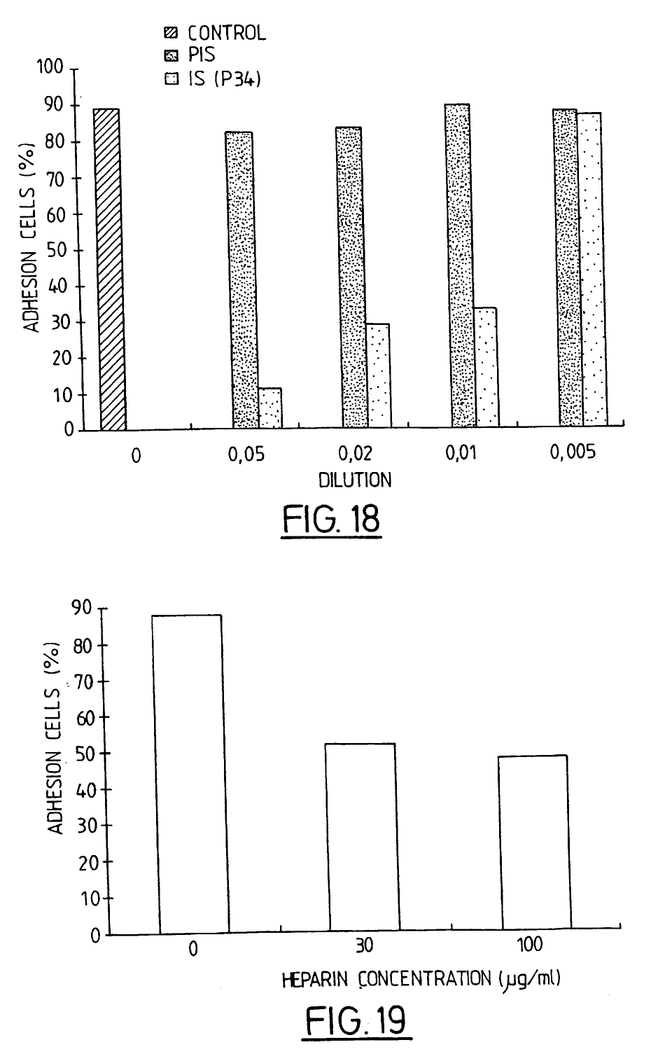

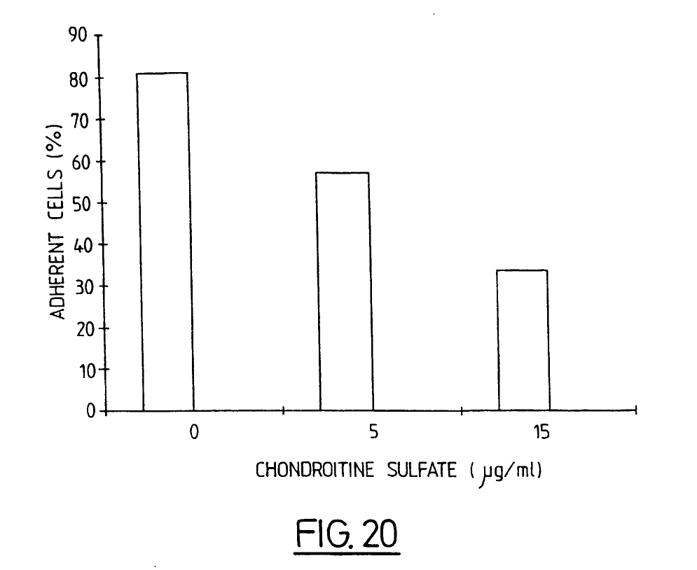

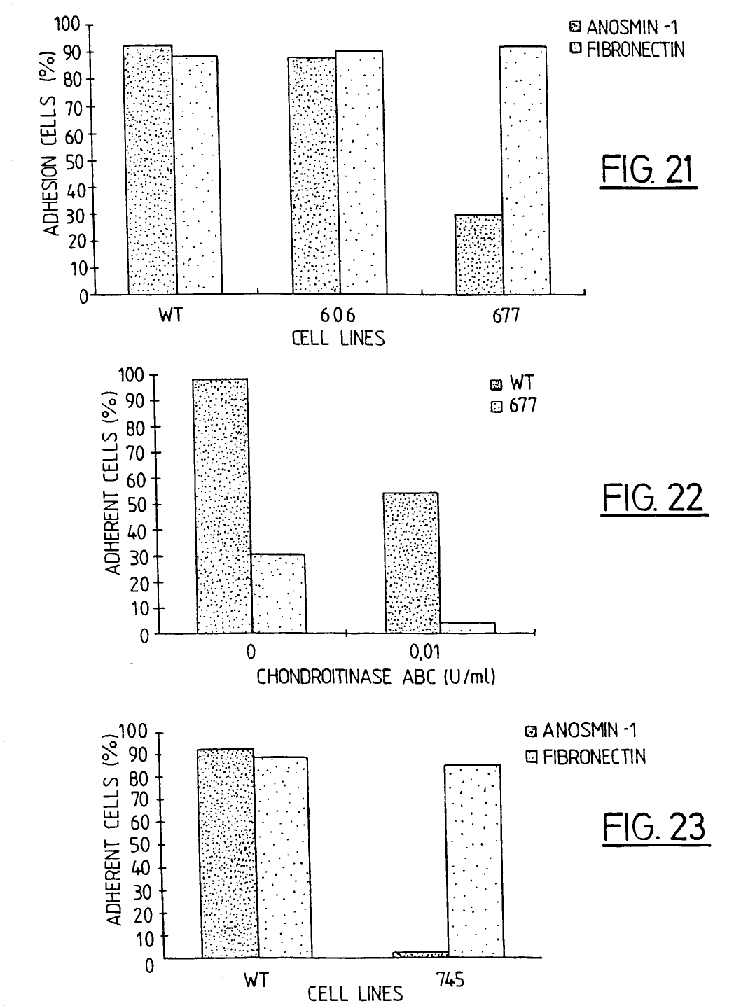

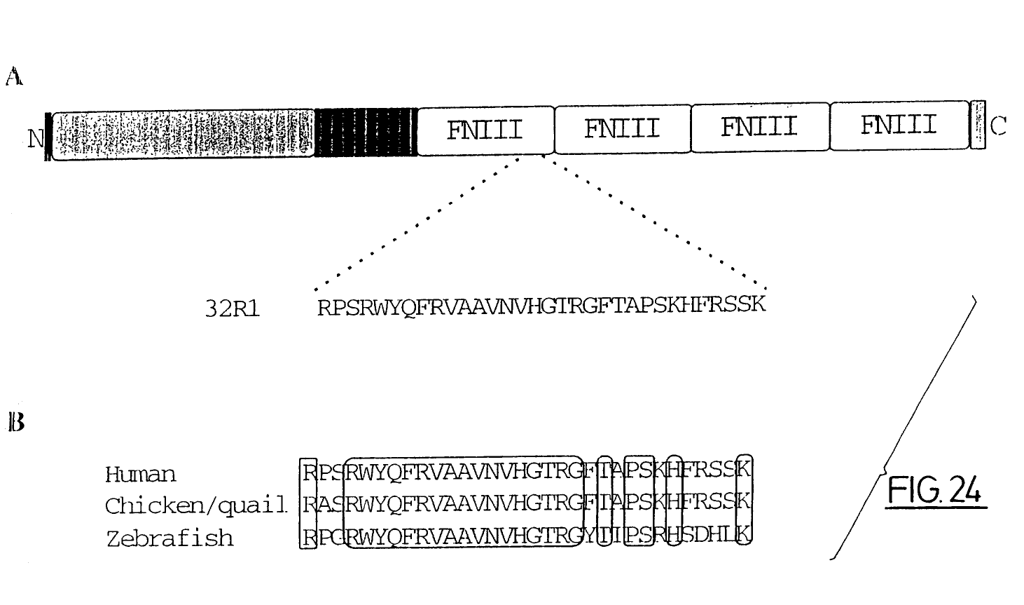

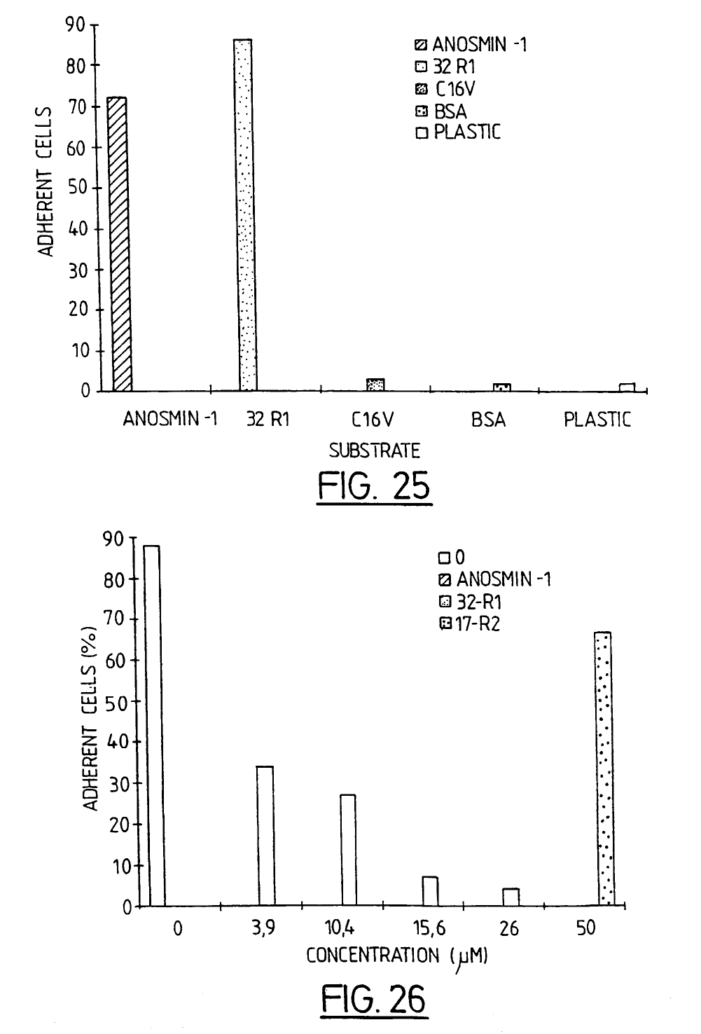

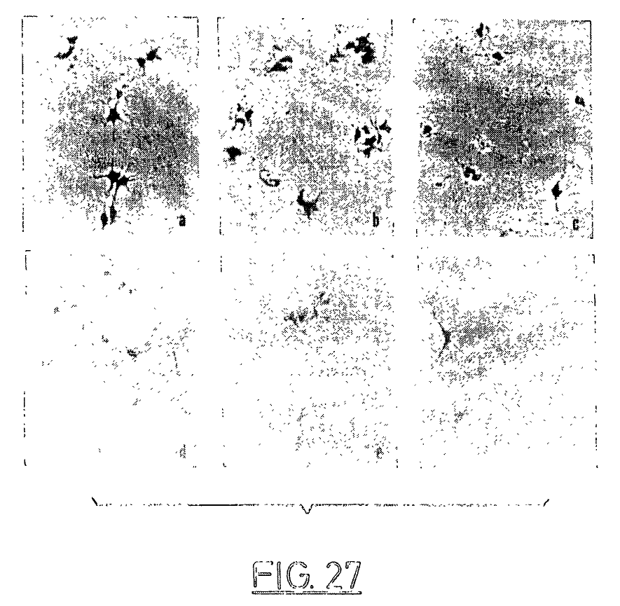

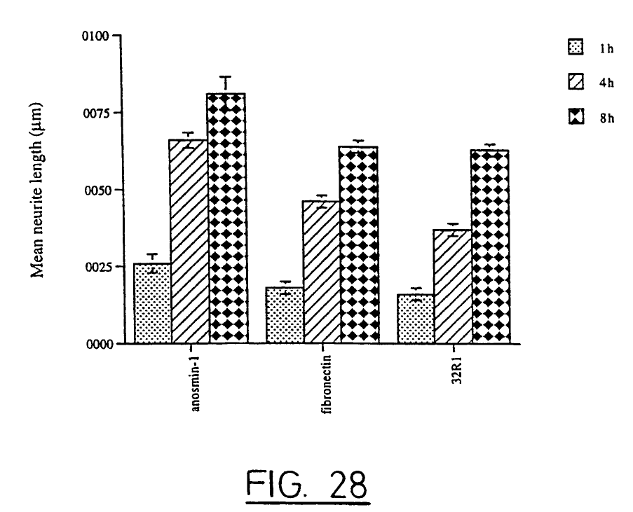

As already mentioned, a particular biologically active part of the KAL protein consists in one or several of the four fibronectin type III repeat of the KAL protein (FIG. 9) alone or in combination one with each other that are obtained by transfection of a procaryotic or an eukaryotic cell, specifically a CHO cell, with the corresponding encoding DNA that has been inserted in a suitable expression vector.

A suitable vector for the expression of the biologically active part of the KAL protein above-defined in baculovirus vector that can be propagated in insect cells and in insect cell lines. A specific suitable host vector system is the pVL 1392/1393 baculovirus transfer vector (Pharmingen) that is used to transfect the SF9 cell line (ATCC No.CRL 1711) which is derived from Spodoptera frugiperda.

Another suitable vector for the expression in bacteria and in particular in E. coli, is the pQE-30 vector (QIAexpress) that allows the production of a recombinant protein containing a 6xHis affinity tag. The 6xHis tag is placed at the C-terminus of the recombinant KAL protein biologically active part which allows a subsequent efficient purification of the recombinant protein by passage onto a Nickel or Copper affinity chromatography column. The Nickel chromatography column may contain the Ni-NTA resin (Porath et al., 1975).

In another embodiment of the therapeutic composition according to the invention, the said composition comprises a polynucleotide coding for the KAL protein or one of its biologically active derivatives in order to perform a gene therapy.

The gene therapy consists in correcting a defect or an anomaly (mutation, aberrant expression etc.) by the introduction of a genetic information in the affected organism. This genetic information may be introduced in vitro in a cell that has been previously extracted from the organism, the modified cell being subsequently reintroduced in the said organism, directly in vivo into the appropriate tissue.

The method for delivering the corresponding protein or peptide to the interior of a cell of a vertebrate in vivo comprises the step of introducing a preparation comprising a pharmaceutically acceptable injectable carrier and a naked polynucleotide operatively coding for the polypeptide is taken up into the interior of the cell and has a pharmaceutical effect at the renal, retinal or the neuronal level of the vertebrate.

In a specific embodiment, the invention provides a pharmaceutical product, comprising a naked polynucleotide operatively coding for the KAL protein or one of its biologically active derivatives, in solution in a physiologically acceptable injectable carrier and suitable for introduction interstitially into a tissue to cause cells of the tissue to express the said protein or polypeptide.

Advantageously, the therapeutic composition containing a naked polynucleotide is administered locally, near the site to be treated.

The polynucleotide operatively coding for the KAL protein or one of its biologically active derivatives is a vector comprising the genomic DNA or the complementary DNA coding for the KAL protein or its protein derivative and a promoter sequence allowing the expression of the genomic DNA or the complementary DNA in the desired vertebrate cells.

The vector component of a therapeutic composition according - to the present invention is advantageously a plasmid, a part of which is of bacterial origin, which carries a bacterial origin of replication and a gene allowing its selection such as an antibiotic resistance gene.

By “vector” according to this specific embodiment of the invention is intended a circular or linear DNA molecule.

This vector may also contain an origin of replication that allows it to replicate in the vertebrate host cell such as an origin of replication from a bovine papillomavirus.

The promoter carried by the said vector is advantageously the cytomegalovirus promoter (CMV). Nevertheless, the promoter may also be any other promoter with the proviso that the said promoter allow an efficient expression of the DNA insert coding for the KAL protein or one of its biologically active derivatives within the host.

Thus, the promoter is selected among the group comprising:

an internal or an endogenous promoter, such as the natural promoter associated with the structural gene coding for KAL; such a promoter may be completed by a regulatory element derived from the vertebrate host, in particular an activator element;

a promoter derived from a cytoskeletal protein gene such as the desmin promoter (Bolmont et al., J. of Submicroscopic cytology and pathology, 1990, 22:117-122; Zhenlin et al., Gene, 1989, 78:243-254).

As a general feature, the promoter may be heterologous to the vertebrate host, but it is advantageously homologous to the vertebrate host.

By a promoter heterologous to the vertebrate host is intended a promoter that is not found naturally in the vertebrate host.

Therapeutic compositions comprising a naked polynucleotide are described in the PCT application No. WO 90/11092 (Vical Inc.) and also in the PCT application No. WO 95/11307 (Institut Pasteur, INSERM, Universite d'Ottawa) as well as in the articles of Tacson et al. (1996, Nature Medicine, -2 (8):888-892) and of Huygen et al. (1996, Nature Medicine, 2(8):893-898).

The therapeutic compositions described above may be administered to the vertebrate host by a local route such as an intramuscular route.

The therapeutic naked polynucleotide according to the present invention may be injected to the host after it has been coupled with compounds that promote the penetration of the therapeutic polynucleotide within the cell or its transport to the cell nucleus. The resulting conjugates may be encapsulated in polymer microparticles as it is described in the PCT application No. WO 94/27238 (Medisorb Technologies International).

In another embodiment, the DNA to be introduced is complexed with DEAE-dextran (Pagano et al., 1967, J. Virol., 1:891) or with nuclear proteins (Kaneda et al., 1989, Science 24:375), with lipids (Feigner et al., 1987, Proc. natl. Acad. Sci., 84:7413) or encapsulated within liposomes (Fraley et al., 1980, J. Biol. Chem., 255:10431).

In another embodiment, the therapeutic polynucleotide may be included in a transfection system comprising polypeptides that promote its penetration within the host cells as it is described in the PCT application WO 95/.10534 (Seikagaku Corporation).

The therapeutic polynucleotide and vector according to the present invention may advantageously be administered in the form of a gel that facilitates their transfection into the cells. Such a gel composition may be a complex of poly-L- Lysine and lactose, as described by Midoux (1993, Nucleic Acids Research, 21:871-878) or also poloxamer 407 as described by Pastore (1994, Circulation, 90:I-517). The therapeutic polynucleotide and vector according to the invention may also be suspended in a buffer solution or be associated with liposomes.

Thus, the therapeutic polynucleotide and vector according to the invention are used to make pharmaceutical compositions for delivering the DNA (genomic DNA or cDNA) coding for the KAL protein or one of its biologically active derivatives at the site of the injection.

The amount of the vector to be injected vary according to the site of injection and also to the kind of disorder to be treated. As an indicative dose, it will be injected between 0.1 and 100 μg of the vector in a patient.

In another embodiment of the therapeutic polynucleotide according to the invention, this polynucleotide may be introduced in vitro in a host cell, preferably in a host cell previously harvested from the patient to be treated and more preferably a somatic cell such as a muscle cell, a renal cell or a neurone. In a subsequent step, the cell that has been transformed with the therapeutic nucleotide coding for the KAL protein or one of its biologically active derivative is implanted back into the patient body in order to deliver the recombinant protein within the body either locally or systemically.

In a preferred embodiment, gene targeting techniques are used to introduce the therapeutic polynucleotide into the host cell. One of the preferred targeting techniques according to the present invention consists in a process for specific replacement, in particular by targeting the KAL protein encoding DNA, called insertion DNA, comprising all or part of the DNA structurally encoding for the KAL protein or one of its biologically active derivatives, when it is recombined with a complementing DNA in order to supply a complete recombinant gene in the genome of the host cell of the patient, characterized in that:

the site of insertion is located in a selected gene, called the recipient gene, containing the complementing DNA encoding the KAL protein or one of its biologically active derivatives and in that

the polynucleotide coding for the KAL protein or one of its biologically active derivatives may comprise:

“flanking sequences” on either side of the DNA to be inserted, respectively homologous to two genomic sequences which are adjacent to the desired insertion site in the recipient gene.

the insertion DNA being heterologous with respect to the recipient gene, and

the flanking sequences being selected from those which constitute the abovementioned complementing DNA and which allow as a result of homologous recombination with corresponding sequences in the recipient gene, the reconstitution of a complete recombinant gene in the genome of the eukaryotic cell.

Such a DNA targeting technique is described in the PCT patent application No. WO 90/11354 (Institut Pasteur).

Such a DNA targeting process makes it possible to insert the therapeutic nucleotide according to the invention behind an endogenous promoter which has the desired functions (for example, specificity of expression in the selected target tissue).

According to this embodiment of the invention, the inserted therapeutic polynucleotide may contain between the flanking sequences and upstream from the open reading frame encoding the KAL protein or one of its biologically active derivatives, a sequence carrying a promoter sequence either homologous or heterologous with respect to the KAL encoding DNA. The insertion DNA may contain in addition, downstream from the open reading frame and still between the flanking sequences, a gene coding for a selection agent, associated with a promoter making possible its expression in the target cell.

According to this embodiment of the present invention, the vector used contains in addition a bacterial origin of replication of the type colE1, pBR322, which makes the clonings and preparation in E. coli possible. A preferred vector is the plasmid pGN described in the PCT application No. WO 90/11354.

Other gene therapy methods than those using homologous recombination may also be used in order to allow the expression of a polynucleotide encoding the KAL protein or one of its biologically active derivatives within a patient's body.

In all the gene therapy methods that may be used according to the present invention, different types of vectors are utilized.

In one specific embodiment, the vector is derived from an adenovirus. Adenoviruses vectors that are suitable according to the gene therapy methods of the present invention are those described by Feldman and Steg (1996, Medicine/Sciences, synthese, 12:47-55) or Ohno et al. (1994, Sciences, 265:781-784) or also in the French patent application No. FR-94 03 151 (Institut Pasteur, Inserm). Another preferred recombinant adenovirus according to this specific embodiment of the present invention is the human adenovirus type 2 or 5 (Ad 2 or Ad 5) or an adenovirus of animal origin (French patent application No. FR-93.05954).

Among the adenoviruses of animal origin it can be cited the adenoviruses of canine (CA V2, strain Manhattan or A26/6 [ATCC VR-800]), bovine, murine (Mavl, Beard et al., 1980, Virology, 75:81) or simian (SAV).

Preferably, the inventors are using recombinant defective adenoviruses that may be prepared following a technique well-known by one of skill in the art, for example as described by Levrero et al., 1991, Gene, 101:195) or by Graham (1984, EMBO J., 3:2917) or in the European patent application No. EP-185.573. Another defective recombinant adenovirus that may be used according to the present invention, as well as a pharmaceutical composition containing such a defective recombinant adenovirus, is described in the PCT application No. WO 95/14785.

In another specific embodiment, the vector is a recombinant retroviral vector, such as the vector described in the PCT application No. WO 92/15676 or the vector described in the PCT application No. WO 94/24298 (Institut Pasteur). The latter recombinant retroviral vector comprises:

a DNA sequence from a provirus that has been modified such that:

the gag, pol and env genes of the provirus DNA has been deleted at least in part in order to obtain a proviral DNA which is incapable of replicate, this DNA not being able to recombine to form a wild virus;

the LTR sequence comprises a deletion in the U3 sequence, such that the mRNA transcription that the LTR controls is significantly reduced, for example at least 10 times, and

the retroviral vector comprises in addition an exogenous nucleotide sequence coding for the KAL protein or one of its biologically active derivatives under the control of an exogenous promoter, for example a constitutive or an inductible promoter.

By exogenous promoter in the recombinant retroviral vector described above is intended a promoter that is exogenous with respect to the retroviral DNA but that may be endogenous or homologous with respect to the KAL protein entire or partial nucleotide coding sequence.

In the case in which the promoter is heterologous with respect to the KAL protein entire or partial nucleotide coding sequence, the promoter is preferably the mouse inductible promoter Mx or a promoter comprising a tetracyclin operator or also a hormone regulated promoter. A preferred constitutive promoter that is used is one of the internal promoters that are active in the resting fibroblasts such the promoter of the phosphoglycerate kinase gene (PGK-1). The PGK-1 promoter is either the mouse promoter or the human promoter such as described by Adra et al. (1987, Gene, 60:65-74). Other constitutive promoters may also be used such that the beta-actin promoter (Kort et al., 1983, Nucleic Acids Research, 11:8287-8301) or the vimentin promoter (Rettlez and Basenga, 1987, Mol. Cell. Biol., 7:1676-1685).

A preferred retroviral vector used according to this specific embodiment of the present invention is derived from the Mo-MuLV retrovirus (WO 94/24298).

In one preferred embodiment, the recombinant retroviral vector carrying the therapeutic nucleotide sequence coding for the KAL protein or one of its biologically active derivatives is used to transform mammalian cells, preferably autologous cells from the mammalian host to be treated, and more preferably autologous fibroblasts from the patient to be treated. The fibroblasts that have been transformed with the retroviral vector according to the invention are reimplanted directly in the patient's body or are seeded in a preformed implant before the introduction of the implant colonized with the transformed fibroblasts within the patient's body. The implant used is advantageously made of a biocompatible carrier allowing the transformed fibroblasts to anchor associated with a compound allowing the gelification of the cells. The biocompatible carrier is either a biological carrier, such as coral or bone powder, or a synthetic carrier, such as synthetic polymer fibres, for example polytetrafluoroethylene fibres.

An implant having the characteristics as defined above is the implant described in the PCT application No. WO 94/24298 (Institut Pasteur).

Another subject of the present invention is a method for screening ligands that bind to the KAL protein.

Such a screening method, in one embodiment, comprises the steps of:

a) Preparing a complex between the KAL protein and a ligand that binds to the KAL protein by a method selected among the followings:

preparing a tissue extract containing the KAL protein putatively bound to a natural ligand;

bringing into contact the purified KAL protein with a solution containing a molecule to be tested as a ligand binding to the KAL protein.

b) visualizing the complex formed between the KAL protein from the tissue extract and the natural ligand of the KAL protein or the complex formed between the purified KAL protein and the molecule to be tested.

For the purpose of the present invention, a ligand means a molecule, such as a protein, a peptide, a hormone, or antibody or a synthetic compound capable of binding to the KAL protein or one of its biologically active derivatives or to modulate the expression of the polynucleotide coding for the KAL protein or coding for one of its biologically active derivatives.

In the first embodiment of the screening procedure wherein a natural ligand of the KAL protein is to be characterized, it is processed as follows:

The tissue putatively containing the KAL protein bound to its natural ligand, for example the cerebellum, olfactory bulbs, tectum or liver from embryos, specifically chicken embryos, are homogenized in 10 mM Hepes, pH 7.4, containing 100 μg/ml PMSF, 200 μg/ml aprotinin and 5 μg/ml Dnase, with a glass-Teflon homogenizer. The homogenate is centrifuged at 1,000 g for 10 minutes; the supernatant is removed and centrifuged at 190,000 g for 30 min at 4° C. The pellet containing the membrane fraction is stored at −20° C. until used.

The cell membrane fractions are incubated first in 0.9% Triton X-100, 0.1% ovalbumin, 5 mM EDTA, 50 mM Tris-HCl, pH 8, with the P34 immune serum (Soussi-Yanicostas et al., 1996) overnight at 4° C., then with Protein G-sepharose (Pharmacia) for 2 hours. Complexes are centrifuged, washed three times in PBS and three times in 50 mM Tris-HCl, pH 8. Then the complexes are dissociated in a dissociating buffer containing SDS in order to dissociate the KAL protein from its bound natural ligand. Immunoprecipitates are analysed by western blot following the technique described by Gershoni and Palade (1983, Anal. Biochem., 131:1-15). The anti-KAL protein monoclonal antibody produced by the hybridoma clone 1-4 was used to detect the KAL protein and a panel of candidate antibodies, for example antibodies directed against different sub-units of integrins are used (at a concentration of 1.5 μg/ml) to identify the ligand that was previously bound to the KAL protein in the tissue extract. IgG peroxidase-conjugated antibody (Bio-Rad, 1/6,000 dilution) is used as second antibody. The blots are revealed by chemiluminescence with the ECL kit (Amersham France).

In a second embodiment of the ligand screening method according to the present invention, a biological sample or a defined molecule to be tested as a putative ligand of the KAL protein is brought into contact with the purified KAL protein, for example the purified recombinant KAL produced by the clone CH KAL 2-3/dl, in order to form a complex between the KAL protein and the putative ligand molecule to be tested. The biological sample may be obtained from a cerebellum or a renal extract, for example.

When the ligand source is a biological sample, the complexes are processed as described above in order to identify and characterize the unknown ligand.

When the putative ligand is a defined known molecule to be tested, the complexes formed between the KAL protein and the molecule to be tested are not dissociated prior to the western blotting in order to allow the detection of the complexes using polyclonal or monoclonal antibodies directed against the KAL protein.

In a particular embodiment of the screening method, the putative ligand is the expression product of a DNA insert contained in a phage vector (Parmley and Smith, Gene, 1988, 73:305-318). According to this particular embodiment, the recombinant phages expressing a protein that binds to the immobilized KAL protein is retained and the complex formed between the KAL protein and the recombinant phage is subsequently immunoprecipitated by a polyclonal or a monoclonal antibody directed against the KAL protein.

According to this particular embodiment, a ligand library is constructed in recombinated phages from human of chicken genomic DNA or cDNA. Once the ligand library in recombinant phages has been constructed, the phase population is brought into contact with the immobilized KAL protein. The preparation of complexes is washed in order to remove the non-specifically bound recombinant phages. The phages that bind specifically to the KAL protein are then eluted by a buffer (acid pH) or immunoprecipitated by the monoclonal antibody produced by the hybridoma anti-KAL, clone 1,4, and this phage population is subsequently amplified by an over-infection of bacteria (for example E. coli). The selection step may be repeated several times, preferably 2-4 times, in order to select the more specific recombinant phage clones. The last step consists in characterizing the protein produced by the selected recombinant phage clones either by expression in infected bacteria and isolation, expressing the phage insert in another host-vector system, or sequencing the insert contained in the selected recombinant phages.

One group of the numerous candidate ligands that may be screened belong to the integrin protein family.

Another subject of the present invention is a method for screening molecules that modulate the expression of the KAL protein. Such a screening method comprises the steps of:

a) cultivating a prokaryotic or an eukaryotic cell that has been transfected with a nucleotide sequence encoding the KAL protein, placed under the control of its own promoter;

b) bringing into contact the cultivated cell with a molecule to be tested;

c) quantifying the expression of the KAL protein.

Using DNA recombinant techniques well known by the one skilled in the art, the KAL protein encoding DNA sequence is inserted into an expression vector, downstream from its promoter sequence, the said promoter sequence being described by Cohen-Salmon et al. (1995, Gene, 164:235-242).

The quantification of the expression of the KAL protein may be realized either at the mRNA level or at the protein level. In the latter case, polyclonal or monoclonal antibodies may be used to quantify the amounts of the KAL protein that have been produced, for example in an ELISA or a RLA assay.

In a preferred embodiment, the quantification of the KAL mRNA is realized by a quantitative PCR amplification of the cDNA obtained by a reverse transcription of the total mRNA of the cultivated KAL-transfected host cell, using a pair of primers specific for KAL of the kind that are described in the PCT application No. WO 93/02267 (Institut Pasteur, HHS).

As an illustrative example, a pair of primers used to quantitate KAL reverse-transcribed mRNA is the following: Primer 1: 5′ CAG CCA ATG GTG CGG CCT CCT GTC C3′Primer 2: 5′ TCC CGG CAG ACA GCG ACT CCGT 3′

The process for determining the quantity of the cDNA corresponding to the KAL mRNA present in the cultivated KAL-transfected cells is characterized in that:

1) a standard DNA fragment, which differs from the KAL cDNA fragment, obtained by the reverse transcription of the KAL-mRNA, but can be amplified with the same oligonucleotide primers is added to the sample to be analyzed containing the KAL-cDNA fragment, the standard DNA fragment and the KAL-cDNA fragment differing in sequence and/or size by not more than approximately 10%, and preferably by not more than 5 nucleotides by strand,

2) the KAL-cDNA fragment and the standard DNA fragment are coamplified with the same oligonucleotide primers, preferably to saturation of the amplification of the KAL-cDNA fragment,

3) to the reaction medium obtained in step 2), there are added:

either two types of labeled oligonucleotide probes which are each specific for the KAL-cDNA fragment and the standard DNA fragment and different from said oligonucleotide primers of step 2), and one of more additional amplification cycle(s) with said labeled oligonucleotide primer(s) is/are performed, so that, during a cycle, after denaturation of the DNA, said labeled oligonucleotide primer(s) hybridize(s) with said fragments at a suitable site in order than a elongation with the DNA polymerase generates labeled DNA fragments of different sizes and/or sequences and/or with different labels according to whether they originate from the DNA fragment of interest or the standard fragment, respectively, and then 4) the initial quantity of KAL-cDNA fragment is determined as being the product of the initial quantity of standard DNA fragment and the ratio of the quantity of amplified KAL-cDNA fragment, which ratio is identical to that of the quantities of the labeled DNA fragments originating from the amplified KAL-cDNA fragment, respectively, obtained in step 3.)

Primers and probes hybridizing with the KAL-cDNA fragment and used in the above-described quantitative PCR amplifications reaction are described in the PCT application No. WO 93/072679 Institut Pasteur, HHS).

More technical details regarding the performing of the quantitative PCR amplification reaction are found in the PCT application No. WO 93/10257 (Institut Pasteur, Inserm).

Materials and Methods

Antibodies

Immunoglobulins from pre-immune and anti-human Kal rabbit sera were purified by affinity chromatography on protein-A sepharose (Pharmacia Biotech., Sweden). Fragments with an antigen-binding site (Fab) were prepared by proteolytic digestion with papain-agarose (Sigman, USA), undigested IgG were eliminated by protein-A sepharose chromatography and Fab were extensively dialyzed against PBS.

Cell Culture

All the culture media, fetal calf serum (FCS) and horse serum were purchased from Life Technologies (France).

The parental CHO cell line and the human KAL-transfected CHO clones (1-1 and 2-3) were cultivated in DMEM medium supplemented with 8% fetal calf serum (PAA, Jacques Boy, France).

Cells from the olfactory neuronal line 13.S.1.24 also named “line 24” (kindly provided by Dr. Astic, Lyon, France) were cultivated in DMEM supplemented with 10% calf serum. Porcine kidney epithelial cells (LLCPK) ATCC NO CRL1392 Mouse GnRH neurons (GT1, subclone 1) Wild-type CHO-K1, mutant CHO-K1-677 (Lidholt K. et al., 1992) and CHO-K1-606 (Bame et al., 1994) cell lines were provided to us by Dr. Esko, the mutant CHO-K1-745 (Esko et al., 1987) cell line was obtained from ATCC. The CHO-K1-677 cells display undetectable levels of heparan-sulfate (HS), but synthesize chondroitin sulfate (CS) at a level 3 times higher than wild-type CHO-K1 cells. The CHO-K1-606 cells express HS and CS at levels similar to those observed in wild-type, but HS is 2 to 3 times less sulfated than in wild-type cells. The CHO-K1-745 cells, deficient in xylosyltransferase, synthesize neither HS nor CS (Esko et al., 1987). Wild-type and mutant CHO-K1 cells were maintained in Ham F12 medium supplemented with 10% fetal calf serum.

Recombinant CHO cell lines. The 2,4 kb EcoRI insert from the Blue script plasmid p85 (Legouis et al., 1991, Cell, 67:423-435) consisting of the entire 2,040 bp coding region of the human KAL cDNA (GenBank accession number M97252), as well as 56 bp and 293 bp of the 5′ and 3′ non coding regions, respectively, was introduced, downstream of the CMV/T7 promoter, into a modified pFR400 vector (Genentech Inc., San Francisco, Calif.), pFRCM, that contains a mouse mutant dihydrofolate reductase (dhfr) cDNA. The above-defined p85B plasmid contains a cDNA having the sequence of FIG. 9 and has been deposited at the CNCM (Collection Nationale de Cultures de Microorganismes) on Sep. 26, 1991 under the accession number No. I-1146. This PFRCM-KAL construct was transfected into dhfr+CHO cells by calcium phosphate precipitation (Wigler et al., 1979, Cell, 16:777-785). CHO cells were cultivated in Dulbecco's modified Eagle's medium (DMEM) supplemented with 8% fetal calf serum 9Jacques Boy, France). Several independent clones producing KALc were obtained by stepwise selection in increasing concentrations of methotrexate (from 0.3 μM to 1 mM) as previously described 9Kaufman and Sharp, 1982, J. Mol. Biol., 159:301-621). Expression of KALc was assessed at each step by immunocytolabeling using a polyclonal antibody that has been prepared against the human KAL protein. Clone CHKAL2-3/d11, which is a subclone of the clone CHO-CAL 2.3 was specifically selected. The parental CHO cell line and the human Kal-transfected CHO clones (1-1 and 2-3) were maintained in DMEM -supplemented with 8% FCS, 50 UI/ml penicillin and 50 μg/ml streptomycin.

Cerebellar cell cultures. Dissociated cell cultures were obtained from Swiss mouse cerebella on postnatal day 5. At this age, granule cells account for up to 90% of the total cell population, glial cells included. Cells were dissociated by combined trituration and trypsinisation, and grown in chemically defined medium DMEM/Ham's F12 (3 vol/1 vol) containing 0.2 mM glutamine, 5 μg/ml insulin, 100 μg/ml tranferrin, 20 nM progesterone, 100 mM purrescine, 30 nM selenium 100 U/ml penicillin and 0.1 mg/ml streptomycin.

Reaggregate cultures of cerebellar neurons from mice on postnatal day-5 were prepared according to Gao et al. (1995). After dissociation, cells were further purified by preplating on a poly-L-Lysine treated (25 μg/ml) substrate for 30 min and plated in uncoated 96-well dishes (5 10⁵ cells/well) in BME plus 10% horse serum, 5% fetal calf serum, 9 mg/ml glucose, 0.3 mg/ml glutamine, 50 U/ml penicillin and 0.1 mg/ml streptomycin. Aggregates (100-200 cells) were harvested after 24 h to be used in coculture experiments.

Parental and transfected CHO cells (clones 1.1 and 2.3) were seeded in Nunc 8-well labtek slides at a density of 10⁴ cells/well. Cells were grown for 24h until confluency and used as monolayer underlying aggregated cerebellar neurons. Cocultures were established by adding approximately 50 aggregates/labtek well, and maintained for 24 h or 48 h in defined medium prior to fixation and immunostaining. Where indicated, pre-immune or anti-KAL Fab fragments at a concentration of 0.2 mg/ml were included for the entire coculture period.

Indirect immunofluorescence. For the visualization of neurons grown on monolayers, cells were fixed with 4% paraformaldehyde in phosphate buffer salline (PBS) for 15 min, permeabilized with methanol/acetone for 2 min, rehydrated in PBS, incubated with anti-GAP 43 antiserum (Williams et al., 1992, J. Cell Biol. 119 p.885-892) diluted (1:500) in PBS containing 3% bovine serum albumin (BSA) for 1 h, rinsed with PBS, incubated with Texas-red conjugated anti-rabbit immunoglobulin (specific for Fc fragment) diluted (1:100) in PSB containing 3% BSA for 1 h. After washing with PBS, cells were mounted in Mowiol (Calbiochem, USA). Recombinant KAL protein expressed by clones 1.1 and 2.3 was labeled with anti-KAL IgG (dilution 10 μg/ml) after cell fixation with 4% paraformaldehyde in PBS for 15 min and using the same immunofluorescent staining procedure.

Production and purification of KAL protein The KAL protein was purified from CHKAL2-3/d11 cells by a three step procedure including two chromatographies. The cells were washed in Ca²⁺- and Mg²-free PBS and incubated for 30 min in DMEM supplemented with 350 mM NaCl. The cell supernatant was supplemented with 0.5% of 3-((3-cholamidopropyl)-dimethylammonio)-1-propane-sulfonate (CHAPS), 50 μg/ml phenylmethylfulfonyl fluoride (PMSF), 100 μg/ml pepstatin and 100 μg/ml leupeptin, and then loaded onto a heparin-Sepharose column (HiTRAP™ Heparin, Pharmacia). NaCl elution fractions were loaded onto an immobilized copper adsorption chromatography column (HiTRAPT™ chelating CU²⁺, Pharmacia) and the protein was eluted as a single peak at 75 mM imidazole.

Adhesion Assay

24-well microtiter plates were coated at 37° C. overnight with 20 μg/ml of laminin, 5μg/ml of KAL in PBS, pH=7.4. The plates were washed twice with PBS and non specific sites were blocked by the addition of 1% BSA in PBS for 1 hour at 37° C. Wells were washed twice with PBS. Cerebellar neurons or PC12 cells were resuspended in DMEM to a final concentration of 10⁶ cells/ml. 500 μl of this suspension was added to each coated well. Cells were also added to control wells that had been coated with BSA alone. Plates were incubated at 37° C. for 90 min in a 5% CO2 humidified atmosphere. The wells were washed gently twice with 0.5 ml PBS. To remove adherent cells from the wells, 0.5 ml of 0.05% trypsin-EDTA were added to each well. After 10 min at 37° C., the 0.5 ml of trypsin-EDTA containing the detached cells were removed and the number of cells was determined by using a cell counter (Coutler, ZM equipped with a Coultronic 256 channelizer).

Each cell adhesion assay was carried out in triplicate. The ration of adherent cells with respect to the total number of cell×100 was determined as the % of cell adhesion.

Adhesion, Spreading and Neurite Growth

Adhesion assays were performed as described in (á compléter) with some modifications. Coating with the various molecules, at the indicated concentrations in 0.5 ml PBS, was performed in plastic 24-well multidishes (Nunclon™) at 37° C. (1 h or overnight).The cells were then washed three times with PBS, coating blocked with 1% heat-inactivated BSA (á compléter) for at least 1 h at 37° C., followed by three PBS washes, before addition of the cells (10⁶ cells per ml in serum-free medium containing 0.1% heat-inactivated BSA). In standard experiments, purified KAL was applied at 3 μg/ml. Other substrates were used as control, fibronectin (20% g/ml) and poly-L-lysine (100 μg/ml).

Cells were incubated for 30 min to 1 h on coated wells. Medium was then aspirated and unbound cells removed by one wash with PBS. The adherent cells were fixed with 4% paraformaldehyde (PFA) in PBS and stained with 0.1% crystal violet. Quantification of the attached cells was performed using a colorimetric method (a completer). Data are expressed as the percentage of adherent cells on each tested substrate with reference to poly-L-lysine. Assays in triplicate were repeated in three independent experiments.

Effect of Divalent Cations

Cells were incubated for 5 min at room temperature in HBSS containing 5 mM EDTA, washed once with HBSS and preincubated for 1 h at 4° C. in HBSS containing 0.1% BSA in the absence or in the presence of Ca⁺⁺ and/or Mg⁺⁺ prior plating.

Inhibition by heparin/heparan sulfate and chondroitin sulfate.

Heparin, heparan sulfate or chondroitin sulfate (up to 1 mg/ml in PBS) was added in KAL-coated-BSA-saturated wells, and allowed to interact for 2 h at 37° C. After three washings with PBS, CHO-K1 or PC12 cells were plated, and incubated as usual.

Chondroitinase Treatment.

Cells from deficient CHO-677 (and the control CHO-K1-XXX) line were preincubated with or without chondroitinase ABC (Sigma, C-2905) at different concentrations (0.01-1 U/ml) in F12 medium, for 15 min at 37° C. Cells were then plated to KAL-coated wells, and incubated for a further 30 min period of time.

Effect of a synthetic peptide corresponding to the first fibronectin type III repeat of human KAL.

A 32 aminoacid peptide (32R1—FIG. 24) was synthesized (Syntiem laboratory). Control peptides were C17 (NH₂-CSLVPTKKKRRKTTDGF-COOH) derived from the second fibronectin type III repeat of human KAL and C16V (NH₂-CGSYANNNRYGRDPPTSV-COOH), derived from the EYA1 protein (a completer). The peptides, at various concentrations (10 to 30 μg/ml), were directly adsorbed on polystyrene microtiter wells (Immulon 4, Nunc). Adhesion assays were performed as described above.

In peptide inhibition assays, cells were preincubated with the peptides at different concentrations (10 to 100 μg/ml) for 1 h at 4° C., then plated to KAL-coated wells, and incubated for a further 30 min period of time.

Spreading and Neurite Growth.

Cells, in serum-free medium containing 0.1% heat inactivated BSA were seeded on KAL- or laminin-coated wells, and maintained for 20 min to 22 h at 37° C. Adherent cells were fixed, stained with toluidine blue, and photographied with an inverted microscope.

Antibodies Inhibition Assays

For inhibition of cell adhesion, 5×10⁵ PC12 cells were deposited on areas previously coated with KAL and with antiserum directed against the human KAL protein at different concentrations and treated as described for adhesion assay. Each inhibition assay was performed three times in three independent experiments.

Heparin Inhibition Assays

PC12 cells (Greene et al., 1076, Proc. Natl. Acad. Sci. USA, 73: 2424-2428) were added to the wells coated with the KAL protein in the presence of different concentrations of heparin and treated as described for adhesion assays. The assays were performed in triplicate.

Competitive Inhibition of KAL-Mediated Adhesion with Fusion Protein

Human serum albumin fusion protein covering the first repeat of fibronectin type III of KAL protein (R1-FNIII) was produced in yeast. The PC12 cells were incubated with different concentrations of R1-FN111, or with Human Serum Albumin (HSA), or with PBS, for 30 min at 37° C. and added to wells which were coated with KAL protein (5 μg/ml) as described above. The assays were performed in triplicate.

Results

It has been hypothesized that the KAL protein mediates cell adhesion because of its structural similarity with well characterized cell adhesion molecules described by Edelman and Crossin, in 1991. In order to test this hypothesis, we examined the ability of the KAL protein coated on a plastic surface to promote adhesion of cerebellar granule neurons and PC12 cells.

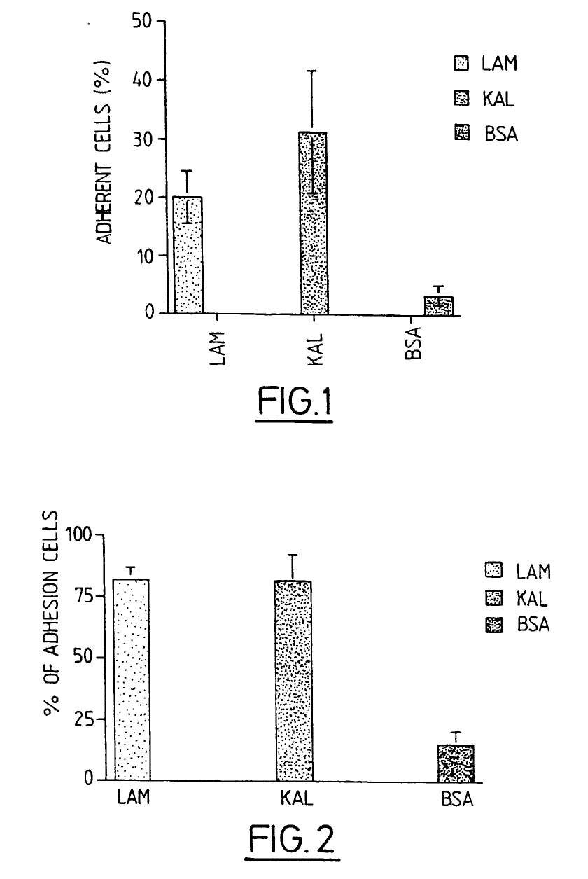

KAL protein isolated from transfected CHO cells was purified by two successive chromatographies on heparin-Sepharose and immobilized copper adsorption columns [Soussi-Yanicostas, 1996 #45] and the purified protein was coated onto Petri dishes. Laminin and bovine serum albumin (BSA) were used as positive and negative controls, respectively. Dissociated mouse cerebellar cells were plated on dishes coated with either KAL protein or laminin, or BSA. After a 90 minute incubation, 80% of the cerebellar neurons were found to adhere to the KAL coated surface. A similar percentage of cell adhesion was observed with laminin-coated dishes. In contrast, no adhesion was detected on BSA Substrate (FIG. 1). Similar results were observed using PC12 cells (FIG. 2). A maximum percentage of cell adhesion was obtained with a concentration of 5 μg/ml of KAL protein (results not shown).

These data suggest that both cerebellar neurons and PC12 cells have the ability to adhere to KAL substrate.

It has been further tested the ability of KAL to promote adhesion in regard to different cell types including both neuronal cells (a rat olfactory neurons cell line (line 24-FIG. 12) (Coronas et al., 1997); mouse GnRH neurons cell line (line GT1-FIG. 13B) (Mellon et al., 1990); P5 cerebellar granule cells (FIG. 14) and rat pheochromocytoma PC12 cells (FIG. 15)), and non neuronal cells (a kidney epithelial cell line (line LLCPK-FIG. 16) (ref) and Chinese Hamster Ovary (CHO) cells (FIG. 17)). The choose of some cell types was made according to the recent data on the localisation of KAL in human fetus. These results have shown that KAL is a component of the basal laminae of in many organs during organogenesis including kidney and is present in meninges which be crossed by olfactory axons and GnRH neurons during development (data not shown). Cell adhesion assays were performed on microtiter wells coated with purified human KAL produced by CHO cells transfected with the human KAL cDNA. The number of adhering cells was measured after 1 hour using a colorimetric method (see Material & Methods). Adhesion to human KAL was compared to adhesion on other substrates; i.e. laminin (LN), poly-lysine (PLL) and bovine serum albumin (BSA). The percentage of cell adhesion observed on PLL substrate was set as 100% and those calculated with the other substrates tested were set in relation to this value. All these cells, olfactory neurons, GnRH neurons, cerebellar granule cells, PC12 cells, epithelial cells and CHO cells were found to adhere to KAL substrate after 1 hour in a manner comparable to that observed on laminin. These cells showing a similar percentage of adhesion to KAL and laminin ranging from 80% to 95% (FIGS. 12-17), except for, cerebellar granule cells that adhere less on laminin (50%) than on KAL (80%) (FIG. 12-17). In contrast, only about 10% of cells of these cells showed adhesion on BSA substrate (FIG. 12A).

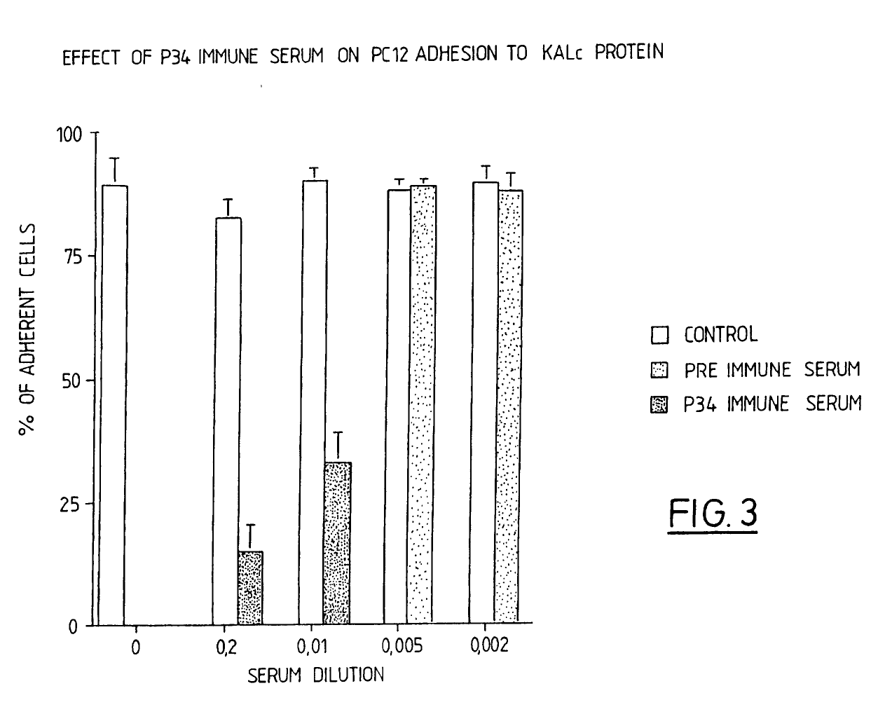

In order to verify that the KAL protein plays a specific role in this cell adhesion, an adhesion assay was performed in the presence of an antiserum directed against the human KAL protein in the culture medium. As shown in FIG. 3, the addition of anti-KAL antibodies inhibits the adhesion of the PC12 cells to KAL-coated dishes. In contrast, the addition of pre-immune serum to the adhesion assay, had no effect on the adhesion of PC12 cells to the KAL protein (FIG. 3).

In order to assess the specificity of the cell adhesion to KAL, adhesion assays were performed on KAL-coated wells preincubated with increasing concentrations of a rabbit immune serum (P34) directed against the purified human KAL. Adhesion of olfactory neurons was prevented by the immune serum P34 in a dose-dependent manner. An inhibition of 80% was observed with an 1/20 dilution of P34 (FIG. 18). A similar inhibition was observed with another immune serum raised against purified KAL (P23) at a 1/20 dilution (data not shown). The inhibition assays was also performed with PC12 cells, P5 cerebellar granule neurons, and CHO cells. Adhesion of these various cell types was 80% inhibited with the same dilution of both immune sera (data not shown).

These results establish that KAL is an efficient adhesion substrate for cells of different phenotypes. This suggests that its-cell surface receptor(s) is (are) widely distributed.

In order to get an insight into the properties of the KAL cell receptor(s), we investigated whether adhesion of PC12 cells on KAL was dependent on the presence of the divalent cations Ca⁺⁺ or Mg⁺⁺. External Ca⁺⁺ and Mg⁺⁺ ions were chelated by incubation of PC12 cells with EDTA for 5 minutes prior plating on KAL-coated microtiter wells (see Materials and Methods). This treatment did not modified the amount of adhering cells therefore, indicating that adhesion of cells to KAL is independent of the presence of Ca⁺⁺ and Mg⁺⁺ cations (data not shown).

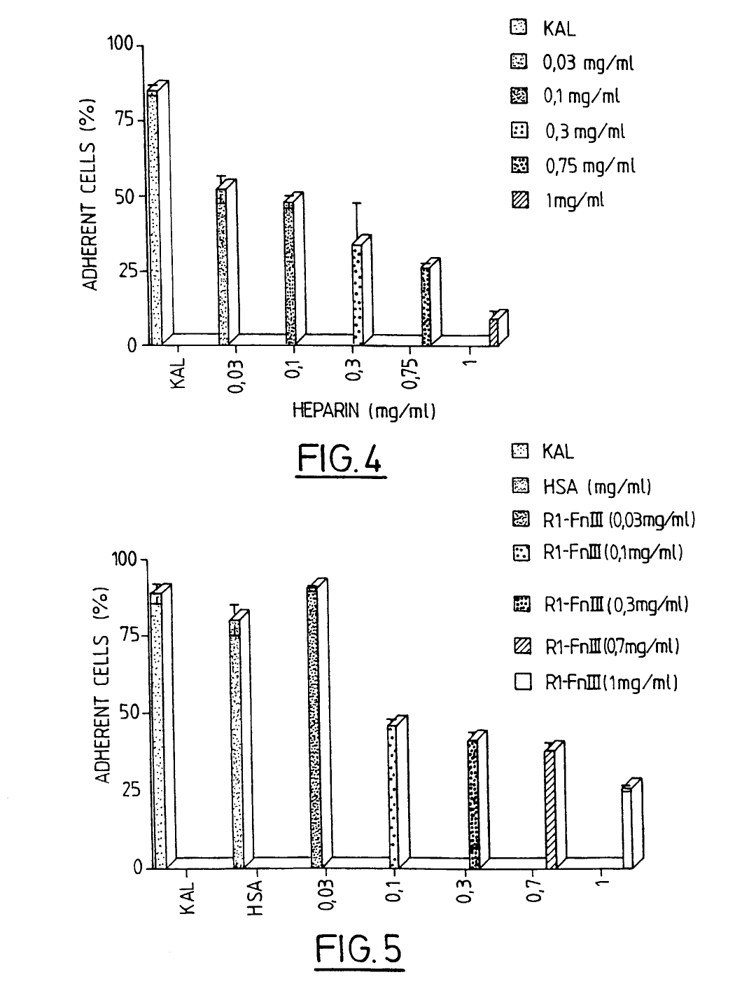

To test whether the interactions of neural cells with KAL protein can be inhibited by addition of soluble glycosaminoglycans, we tested the ability of PC12 cells to adhere to KAL substrates in the presence of heparin. We observed that adhesion of PC12 cells to KAL protein was inhibited from 0.03 mg/ml of heparin (FIG. 4). These results suggest that heparan-sulfateproteoglycans may be involved in the PC12 cell adhesion to KAL protein.

Heparan-sulfate (HSPG) and chondroitin-sulfate proteoglycans (CSPG) are involved in cell adhesion to KAL The inventors have further tested whether cell adhesion to KAL is mediated by cell surface proteoglycans. CHO cells and mutants derived from this cell line were used in several experimental approaches to check whether heparin/heparan-sulfate (HS) or chondroitin-sulfate (CS) interfere with cell adhesion to KAL.

Microtiter wells coated with fixed amount of KAL were incubated with increasing concentrations of HS or CS prior to plating of CHO-K1 cells and the percentage of adherent cells was determined as previously (see Materials and Methods). Heparin (100 μg/ml) induced a significant inhibition of cell adhesion on KAL (approximately 50% of inhibition) (FIG. 19). Similarly, pretreatment with chondroitin-sulfate ABC (15 μg/ml) induced a 50% inhibition of the CHO-K1 cells adhesion (FIG. 20). Similar inhibitory effects (50% of inhibition) of HS or CS on cell adhesion were observed with PC12 cells (data not shown).

In order to further examine the role of HSPG and CSPG in cell adhesion to KAL, adhesion assays were performed with a mutant CHO cell lines deficient in different steps of glycosaminoglycan biosynthesis (Esko et al., 1988). We first examined adherent properties of the CHO-677 cell line which displays undetectable levels of HSPG but overexpresses CSPG (a completer). CHO-677 cells showed about 70% decrease of adhesion to KAL compared to wild-type CHO-K1 cells (FIG. 21). As previously described (Le Baron et al., 1988), when tested on fibronectin as an adhesion substrate both CHO-K1 and CHO-677 cell types showed similar percentages and kinetics of adhesion (FIG. 21). To determine whether CSPG was involved in the adhesion of CHO-677 cells to KAL substrate, these mutant cells were treated by increasing concentrations of chondroitinase ABC (an enzyme degradating chondroitin sulfate ABC) prior plating. This enzymatic treatment resulted in a virtually complete inhibition of adhesion of CHO-677 cells to KAL (FIG. 22), but did not affect adhesion of these mutant cell types on a fibronectin substrate (data not shown). This indicated that chondrotinase ABC treatment did not induce a non-specific inhibitory effect on cell adhesion. Consistent with the role of the chondroitin sulfate in adhesion to KAL, when wild-type CHO-K1 cells were treated by chondroitinase ABC, a decrease of about 50% of the adhesion on KAL was observed (FIG. 22). To confirm that both HSPG and CSPG are involved in cell adhesion to KAL, the adhesive properties of the mutant CHO-745 cell line, deficient in both HSPG and CSPG (a completer) were tested. No adhesion of these cells on KAL could be detected (FIG. 23). In addition, we tested the adhesion properties of mutant cell line CHO-606, expressing HS and CS at similar level as CHO-K1, but which HS is 2 to 3 times less sulfated than wild type. These cells CHO-606 were able to adhere to KAL in a manner comparable to that found in wild-type (CHO-K1) (FIG. 21). This result indicates that this degree of sulfatation of heparin is not significantly affect the ability of CHO-606 to bind KAL.

To investigate the involvement of different domains of KAL protein in PC12 cell adhesion, we produced a human serum albumin fusion protein containing the first repeat of fibronectin type III of the KAL protein (R1-FNIII) in yeast, corresponding, from N-terminal end to C-terminal end, to the aminoacid sequence beginning at the aminoacid at position 182 from the sequence of FIG. 9 and ending at the aminoacid at position 286 from the sequence of FIG. 9. Increasing concentrations of R1-FNIII were incubated with PC12 cells for 30 min at 37° C. before adhesion assays on KAL protein. We observed that R1-FNIII perturbs partially the adhesion of PC12 cells to KAL protein (FIG. 5).

In summary, the cell adhesion assays demonstrated that the KAL protein contains binding sites for molecules present at the cell surface of both cerebellar neurons and PC12 cells. The adhesion of neural cells to KAL protein may depend on glycosaminoglycans. The first fibronectin type III domain of the KAL protein partially account for the binding activity of the molecule.

Determination of the KAL region mediating adhesion Sequence comparaison between human (Legouis et al., 1991), chicken/quail (Legouis et al., 1993;1994) and Zebrafish KAL genes, pointed out the extreme conservation of repeat 1 and in particular of two β sheets among the seven constituting this domain (FIG. 24). These observations prompted us to test the putative role of this 32 amino acids sequence in cell adhesion to KAL.

A corresponding synthetic peptide (32R1) was coated to microtiter wells and its adhesive properties toward wild-type CHO-K1, PC12 cells and olfactory neurons were tested. Two other peptides were tested as a control; i.e. an unrelated 16 amino acids peptide (C16V) and a 17 amino acids peptide corresponding to a part of the second fibronectin type III repeat of human KAL (17R2). The percentage of olfactory neurons (line 24) adherent on 32R1 substrate was not significant to that observed with complete KAL (70% and 80% respectively for KAL and 32R1) (FIG. 25). The same results were obtained with PC12 cells and CHO cells (data not shown). In contrast, the cells displayed no adhesion on 17R2 and C16V (FIG. 25).

To further document these results, we tested the ability of 32R1 to inhibit adhesion of olfactory neurons (line 24) to an KAL substrate. Preincubation of olfactory neurons (line 24) with increasing concentrations of 32R1 showed an inhibition of these cells to KAL in a concentration-dependent manner (FIG. 26). A concentration of 26 μM of peptide gives a complete inibition of the adhesion to KAL substrate (FIG. 26). Similar results were also obtained when PC12 or CHO-K1 cells were preincubated with 32R1. In contrast, preincubation of the olfactory neurons line with control 17R2 (50 μM) peptide had no effect on adhesion to KAL (FIG. 26). Adhesion of these cells to fibronectin and laminin substrates upon preincubation with 32R1 had no effect. Altogether, these data indicated that 32R1 sequence on KAL is involved in adhesion to cells.

The Purified KAL Protein is a Permissive Substrate for Neurite Outgrowth of Cerebellar Neurons.

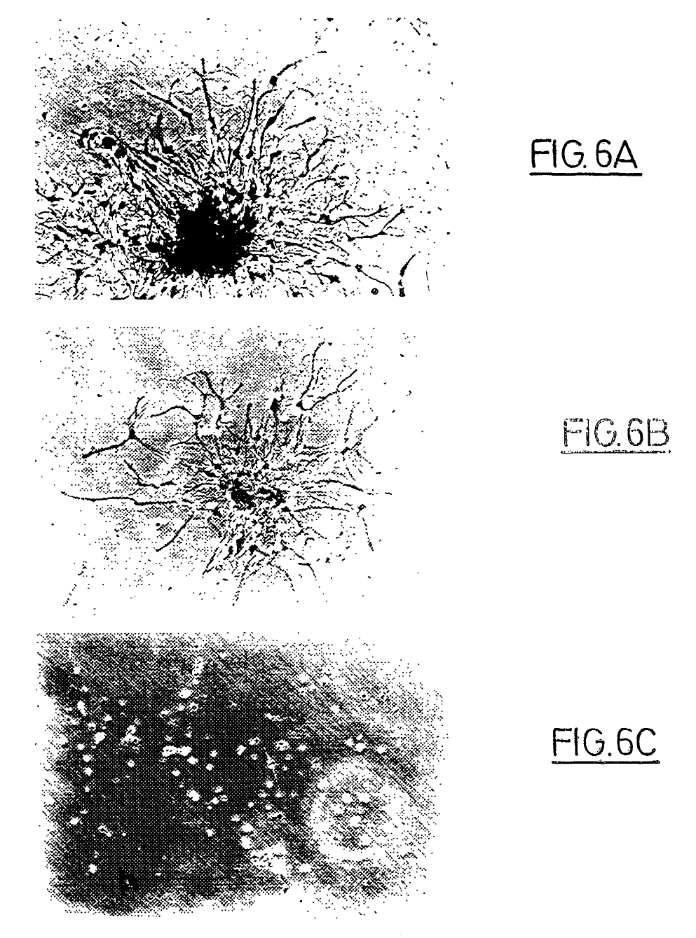

In order to determine the role of purified KAL protein on neurite outgrowth, we used granule cell aggregates as a model, prepared as described in the Materials and Methods section. Cerebellar granule neurons were seeded on surfaces that had been coated with KAL protein. Polylysine and bovine serum albumin (BSA) were used as positive and negative controls respectively. When aggregates were cultured for 48 hours on KAL protein, neurons remained tightly aggregated and displayed a large halo of neuritic processes (FIG. 6A). A similar observation was obtained on the polylysine-coated surface (FIG. 6B). In contrast, no neuronal survival was observed on the BSA-coated surface (FIG. 6C).

These results show that the KAL protein is a permissive substrate for survival and neurite outgrowth of cerebellar granule neurons.

KAL Immunofluorescencestaining at the Surface of Transfected CHO Cells

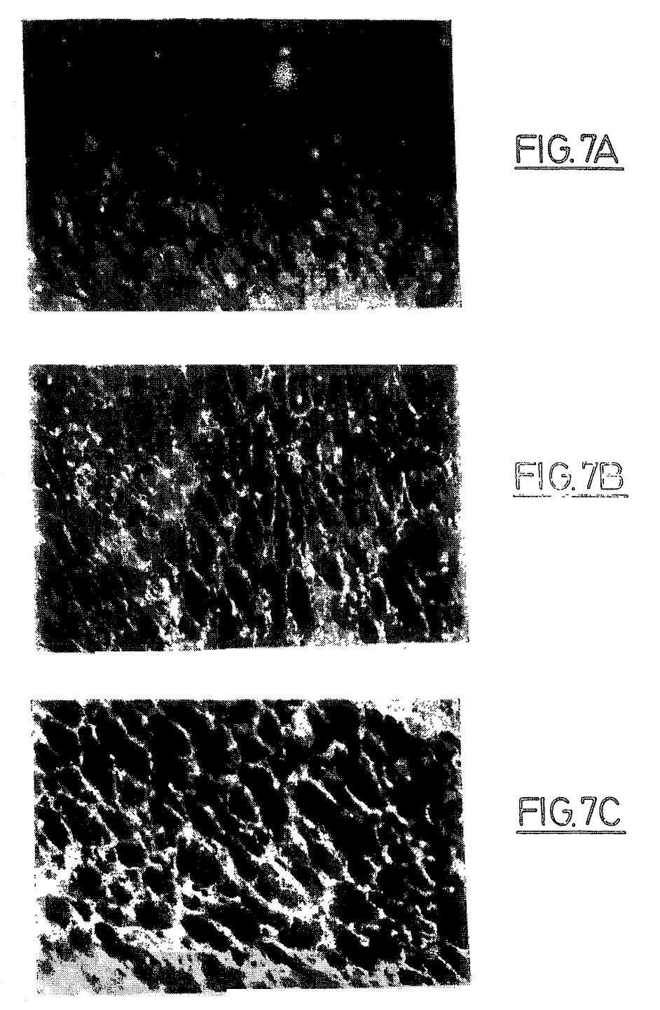

The different human KAL-expressing CHO cell lines were labeled by indirect immunofluorescence using an antiserum directed against the human KAL gene product. Large amounts of the KAL protein were observed at the cell surface of clonal KAL transfected cell lines 1-1 and 2-3 (FIG. 7).

Induction of Neurite Fasciculation from Granule Cell Aggregates by KAL-Expressing Cells

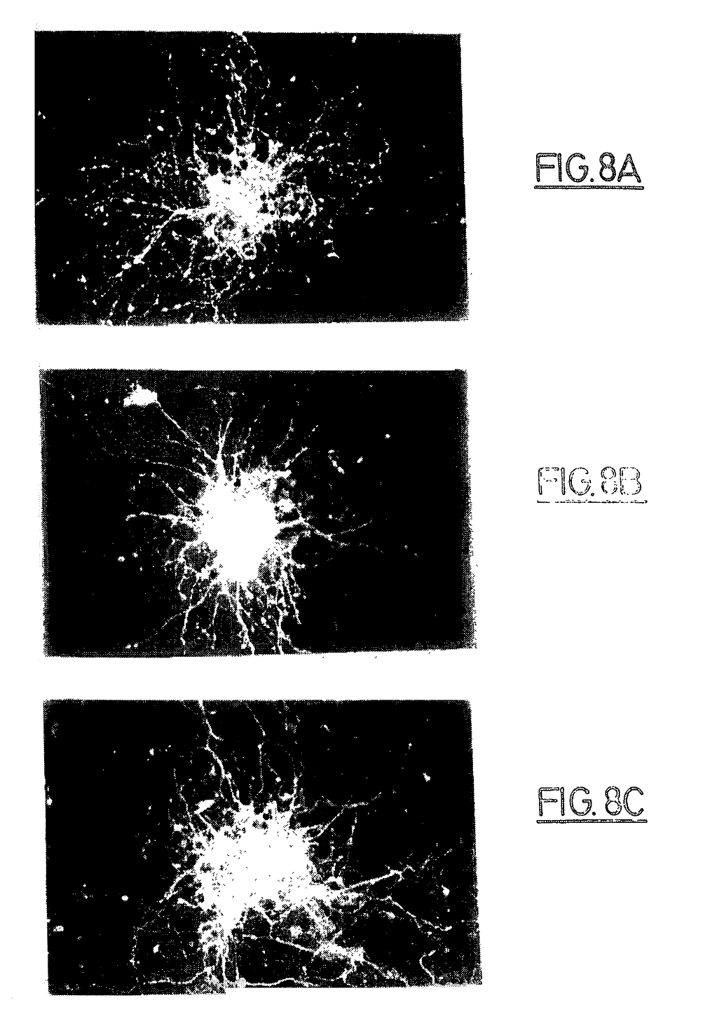

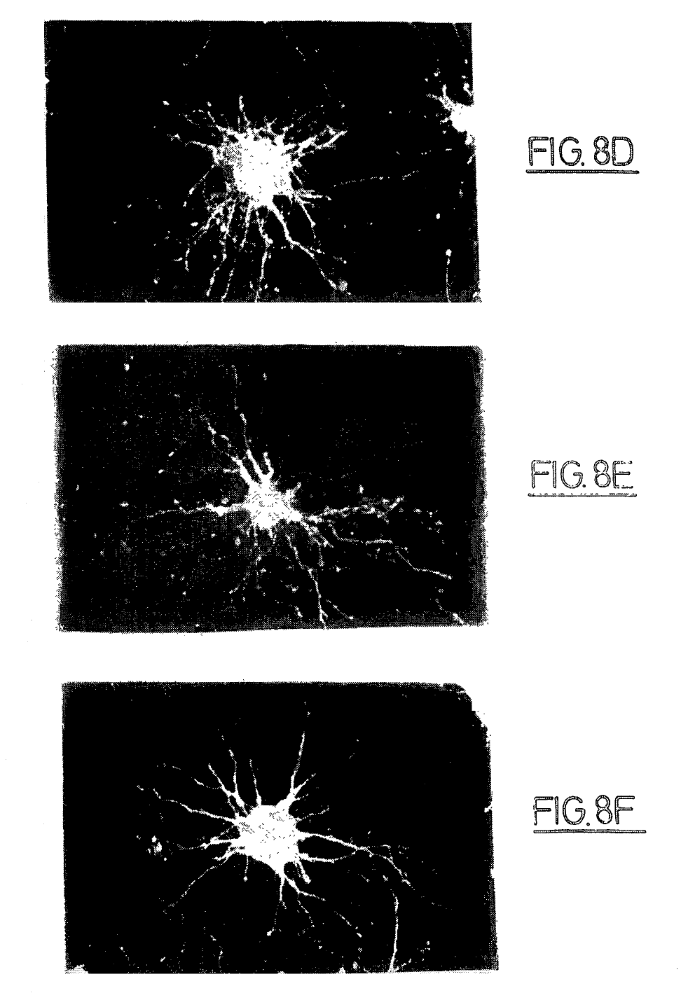

Granule cell aggregates from post-natal day-5 mice were grown in defined medium onto monolayers of CHO cells. After 24 h of coculture, aggregates had produced long, sinuous, and unfasciculated processes onto control cells (FIGS. 8A and 9A) By contrast, aggregates grown onto KAL-expressing cells displayed short, radial and highly fasciculated neurites (FIGS. 8B and 9B). To ensure that this effect was not an artifact of one particular KAL-expressing cell line, two independent clones (1-1 and 2-3) were tested. They were producing equivalent amounts of the transfected protein as assayed by Western blot. These two clones exhibited the same ability to both fasciculate and reduce length of the neuritic processes growing from granule cell aggregates (FIGS. 8D and F).

Antibody Reversal of KAL-Induced Neurite Fasciculation from Granule Cell Aggregates

In order to demonstrate the specificity of Kal's effect on fasciculation and growth inhibition of neurites, anti-KAL fragments (0.2 mg/ml) were included during the entire time of coculture of KAL-expressing cells and granule cell aggregates. KAL-expressing monolayers displayed intense staining with anti-KAL Fab as revealed with Texas-red conjugated IgG specific anti-rabbit antibody (same as FIG. 7B and C, not shown). Both antibodies directed against human KAL and the neuronal marker GAP-43 have been raised in rabbit. Thus, to avoid monolayer staining, neurons were visualized using anti-GAP-43 and Fc-specific Texas red conjugated anti-rabbit antibody.

In the presence of anti-KAL Fab bound to the KAL-expressing cell monolayers, granule cell aggregates showed long and defasciculated neurites (FIGS. 8C, E). Some long neurites were induced to grow circumferentially instead of radially (FIG. 8C). The presence of pre-immune Fab had no effect on the fasciculation and growth inhibition of neurites observed on KAL-expressing CHO cells.

KAL Promotes Neurite Outgrowth from Olfactory Neurons.

Since it has been proposed that the X-linked form of the Kallmann syndrome results from a defect in the embryonic migration of olfactory axons and GnRH neurons, we studied the effect of KAL protein on neurite growth of a 13.S.1.24 line derived from rat olfactory epithelium (Coronas et al., 1997). After differentiation in vitro, this cell line expresses a marker characteristic of olfactory neurons, olfactory marker protein (OMP) (Coronas et al., 1997). The effect of purified KAL protein on neurite growth of olfactory neurons (line 24) was compared to that observed on fibronectin and peptide 32R1. After one hour, cells were well spreaded on KAL protein, fibronectin and peptide 32R1 and neurites were observed in all three cases (FIGS. 27A, B, C); the mean lengths per olfactory neuron were 66 (±2,5) μm, 46 (±4) μm and 32 (±4) μm respectively (FIG. 28). Eight hours after plating, in all these substrates, many cell bodies displayed several neurites (FIGS. 27, C, D), but neurite elongation was stimulated by 30% on KAL protein by comparison with fibronectin and peptide 32R1 (FIG. 28) (the meaning lengths were 81 (±5)μm, 63 (±2,5) μm and 63 (±2)μm, respectively) (FIG. 28).

Characterization of the Cell Adhesion and Neurite Growth Properties of KAL Allows to Put Forward Several Hypotheses Regarding the Functions of this Protein During Development.

According to the results presented in the instant specification that have shown that KAL is a component of the basal laminae of epithelium of many structures including kidney, intestine, respiratory and cardiovascular systems. KAL is colocalized with laminin in basal laminae of epithelium during fetal development in human (Data not shown). Consistently, it has been shown that kidney epithelial cells adhere to KAL. During kidney organogenesis, KAL can mediate stable adhesion that retain cells at the basal membrane, probably in association with -other extracellular matrix proteins such as laminin. This could explain the fact that Kallmann's syndrome in human is sometime associated with renal aplasia.

During later stages of development in chick, KAL is almost restricted to definite neuronal populations in the central nervous system (striatal, retinal, tectal and cerebral neurons), most of which still express the gene after hatching. During these stages, this adhesion molecule may provide a stabilizing role for the maintenance of the structure of fully differenciated tissues. It has been shown that attachment of cells to ECM is necessary for maintenance of tissue integrity. Importance of these cell-ECM interactions is underscored by the phenotypic consequences of many genetic and autoimmune diseases that disturb cell adhesion to ECM in human.

With regard to development of olfactory system, KAL could be involved successively in several processes and hypotheses can be put forward in order to explain the mechanisms leading to GnRH deficiency and Anosmia in KS patients. During early stages in human and chick embryos; (Data not shown) the KALc gene and KAL are expressed in the telencephalic presumptive areas of olfactory bulbs suggesting that KAL may be involved in the morphogenesis of this structure, that probably requires cell-ECM interactions. During the course of development, KAL could play a stabilizating role in mitrales cells as well as being involved in interactions between axons of olfactory neurons and mitral cells neurites (at least in chick).

The inventors data show that KAL mediates adhesion of olfactory neurons (FIG. 12) and induce neurite growth of these neurons (FIG. 22). Our hypothesis is that during brain development in human, KAL directly or indirectly induces outhgrowth of olfactory axons towards the olfactory bulb. Actually, in a KS fetus, it has been shown that migration of olfactory axons and GnRH neurons were arrested within meninges between the cribriform plate and the forebrain. Interestingly, a recent study in our laboratory has shown that in human fetus (5 to 6 weeks of gestation), KAL is expressed in meninges (Data not shown). The absence of KAL in meninges would block the extension of olfactory axons toward olfactory bulbs at the level of meninges. As a consequence, olfactory axons cannot connect dendrites of mitrale cells in the olfactory bulbs. The absence of these connections would explain anosmia in KS patients. With regard to GnRH neurons, since these neurons have been shown to migrate along olfactory nerves, the arrest of extension of olfactory axons at the level of meninges would, as a consequence, block migration of GnRH neurons toward hypothalamus. This could explain the hypogonadism observed in KS patients.

### REFERENCES

Ballabio, Bruce, H. M. (1959). An exteroceptive block to pregnancy in the mouse. Nature 184, 105.

BrŸmmendorf, T., and Rathjen, F. G. (1993). Axonal glycoproteins with immunoglobulin-and fibronectin type III related domains in vertebrates: structural features, binding activities, and signal transduction. J. Neurochem. 61, 1207-1219.

Brummendorf, T. Wolff, J. M., Frank, R., and Rathjen, F. G. (1989). Neural cell recognition molecule F11:homology with fibroectin type III and immunoglobulin type C domains. Neuron 2, 1351-61.

Burgoon, M. P., Grumer, M., Mauro, V., Edelman, G. M., and Cunningham, B. A. (1991). Structure of the chicken nuron-glia cell adhesion molecule, Ng-CAM: origin of the polypeptides and relation to the Ig superfamily, J. Cell. Biol. 112, 1017-29.

Dandekar, A. M., Robinson, E. A., Appella, E., and Qasba, P. K. 91982). Complete sequene analysis of cDNA clones encoding rat whey phosphoprotien: homology to a protease inhibitor. Proc. Natl. Acad. Sci. USA 79, 3987-3991.

De Morsier, G. (1954). Etudes sur les dysraphies cr%nio-enc phaliques. Schweiz Arch. Neurol. psychiat. 74, 309-361.

del Castillo, I., Cohen-Salmon, M., Blanchard, S., Lutfalla, G., and petit, C. (1992). Structure of the X-linked Kallmann syndrome gene and its homologous pseudogene on the Y chromosome. Nature Genet. 2, 305-310.

Edelman, G. M., and Crossin, K. L. (1991). Cell adhesion molecules: implications for a molecular histology. Annu. Rev. Biochem. 60, 155-190.

Engel, J. (1991). Common structural motifs in proteins of the extracellular matrix. Curr. Opinion in Cell Biol. 3, 779-785.

Branco, B., Guioli, S., Pragliola, A., incerti, B., Bardoni, B., Tonlorenzi, R., Carrozzo, R., Maestrini, E. Pieretti, M., Taillon-Miler, P., Brown, C. J., Willard H. Fr., lawrence, C., Persico, M. G., Camerino, G., and Ballaio, A. (1991). A gene deleted in Kallmann's syndrome shares homology with neural cell adhesion and axonal path-finding molecules. Nature 353, 529-536.

Furley, A. J., Morton, S. B., Manalo, D., Karagogeos, D., Dodd, J., and Jessell, T. M. (1990). THe axonal glycoprotein TAG-1 is an immunoglobulin superfamily member wtih neuite outgrowth-promoting activity. Cell 61, 157-70.

Gao, W. Q., Zheng, J. L., and Karihaleo, M. (1995). Neurotrophin-4/5 9NT-4/5) and brain-derived neurotrophic factor (BDNF0 act at later stages of cerebellar granule cell differentiation. J. Neurosci 15, 2656-67.

Gennarini, G., Cibelli, G., Rougon, G., Mattei, M. G., and Goridis, C. (1989). The mouse neuronal cell surface protein F3: a phosphatidylinositol-anchored member of the immunoglobulin superfamily related to chicken contactin. J. Cell. Biol. 109, 775-88.

Gotz, B., Scholze, A., Clement, A., Joester, A., Schutte, K., Wigger, F., Frank R., Spiess, E., Ekblom, P., and Faissner, A. (1996). Tenascin-C contains distinct adhesive,anti-adhesive, and neurite outgrowth promoting sites for neurons. j. Cell. Biol. 132, 681-99.

Greene, L. A., and Tischler, A. S. 91976), Establishment of a noradrenergic clonal line of rat adrenal pheochromocytoma cells which respond to nerve growth factor. proc. Natl Acad Grumet, M., Mauro, V., Burgoon, M. P., Edelman, G. M., and Cunningham, B. A. (1991). Structure of a new nervous system glycoprotein, Nr-CAM, and its relationship to subgroups of neural cell adhesion molecules. J. Cell. Biol. 113, 1399-412.

Hardelin, J.-P., Levilliers, J., del Castillo, I., Cohen-Salmon, M., Legouis, R., Blanchard, S., Compain, S., Bouloux, P., Kirk, J., Moraine, C., Chaussain, J.-L., Weissenbach, J., and Petit, C. (1992). X chromosome-linked Kallmann syndrome: Stop mutations validate the candidate gene. Proc. Natl. Acad. Sci. USA 89, 8190-8194.

Hardelin, J.-P., and Petit, C. (1995). A molecular approach to the pathophysiology of the X chromoxome-linked kallmann's syndrome. In Genetic and Molecular Biological Aspects of Endocrine Disease, R. V. Thakker, ed. (London: Bailli re Tindall), pp. 489-507.

Hennighausen, L. G., and Sippel, A. E. (1982). Mouse whey acidic protein is a novel member of the family of “four-disulfide core” proteins. Nucl. Acids Res. 10, 2677-2684.

Kallmann, F. J., Schoenfeld, W. A., and Barrera, S. E. (1944). The genetic aspects of primary eunochoidism. Am. J. Mental Deficiency XLVIII, 203-236.

Kato, I., and Tominaga, N. (1979). Trypsin-subtilisin inhibitor from red sea turtel eggwhite consits of two tandem domains, one KYnitz, one of a new family. Fed. Proc. 38, 832 9abstract).

Kayyem, J. F., Roman, J. M., de la Rosa, E. J., Schwarz, U., and Dreyer, W. J. (1992). Bravo/Nr-CAM is closely related to the cell adhesion molecules Li and Ng-CAM and has a similar heterodimer structure. J. Cell Biol. 118, 1259-70.

Legouis, R., Cohen-Salmon, M., del Castillo, I., Levilliers, J., Capy, L., Mornon, J.- P., and Petit, C. (1993). characterization of the chicken and quail homologues of the human gene responsible for the X-linked Kallmann syndrome. Genomics 17, 516-518.

Legouis, R., Hardelin, J.-P., Levilliers, J., Claverie, J.-M., Compain, S., Wunderle, V., Millasseau, P., Le Paslier, D., Cohert, D., Caterina, D., Bougueleret, L., Delemarre-Van de Waal, H., Lutfalla, G., Weissenbach, J., and Petit, C. (1991). The candidate gene for the X-linked Kallmann syndrome encodes a protein related to adhesion molecules. Cell 67, 423-435.

Legouis, R., Hardelin, J.-P., Petit, C., and Ayer-Le Li vre, C. (1994). Early expression of the KAL gene during emmbryonic development of the chick. Anat. Embryol. 190, 549-562.

McClintock, M. (1971). Menstrual synchrony and suppression. Nature 229, 244.

Moos, M., Tacke, R., Scherer, H., Teplow, D., Fruh, K., and Schachner, M. (1988). Neural adhesion molecule L1 as a member of the immunoglobulin superfamily with binding domains similir to fironectin. nature 334, 701-3.

Murakami (1994).

Murakami, S., Kukuyama, S., and Arai, Y. (1992). The origin of theluteinizing hormone-releasing hormone (LHRH) neurons in newts (Cynops pyrrhogaster): the effect of olfactory placode ablation. Cell Tussue Res. 269, 21-27.

Murakami, S.,, Seki, T., Wakabayashi, K., and Arai, Y. (1991). The ontogeny of luteinizing hormone-releasing hormone (LHRH) producing neurons int he chick embroyo: possible evidence for migrating LHRH neutrons from the olfactory epithelium expressing a highly polysialyated neural cell adhesion molecule. Neurosci. Res. 12, 421-431.

Naftolin, F., Harris, G. W., and Bobsow, M. (1971). Effect of purified luteinizing hormone releasing factor of normal and hypogonadotropic anosmic men. nature 232, 496-97.

Norenberg, U., Hubert, M., Brummendorf, T., Tarnok, A., and Rathjen, F. G. (1995). Characterization of functional domains of the tenascin-R (restrictin) polypeptide: cell attachment site, binding with F11, and enhancement of F11-mediated neurite outgrowth by tenascin-R. J. Cell. Biol. 130, 473-84.

Norgren (1991).

Ronnekleiv, O. K., and Resko, J. A. (1990). Ontogeny of gonadotropin-releasing hormone-containing neurons in early fetal development of Rhesus macaques: Endocrinology 126, 498-511.

Rugarli, E. I., Lutz, B., Kuratani, S. C., Wawersik, S., Borsani, G., Ballabio, A., and Eichele, G. (1993). Expression pattern of the Kallmann syndrome gene in the olfactory system suggests a role in neuronal targeting. Nature Genet. 4, 19-26.

Schwanzel-Fukuda, M., Abrahams, S., Crossin, K. L., Edelman, G. M., and Pfaff, D. W. (1992). Immunocytochemicall demonstration of neural cell adhesion molecule (NCAM) along the migration route of lateinizing hormone-releasing hormone (LHRH) neurons in mice. J. Comp. Neurol. 321, 1-18.

Schwanzel-Fukuda, M., Bick, D., and Pfaff, D. W. (1989). Luteinizing hormone-releasing hormone (LHRH) -expressing cells do not migrate normally in an inherited hypogonadal (Kallmann) syndrome. Mol. Brain Res. 6, 311-326.

Schwanzel-fukuda, M., and Pfaff, D. W. (1989). Origin of lutinizing hormone-releasing hormone neurons. Nature 338, 161-164.

Schwanzel-fukuda, M., and Pfaff, D. W. (1995). The structure and function of the nervus terminalis. In The handbook of clinical olfaction and teast, R. L. Doty, ed. 9new York: Dekker), pp. 835-864

SeemŸller, U., Arnhold, M., Fritz, H., Wiedenmann, K., Machleidt, W., Heinzel, R., Appelhans, H., Gassen, H.-G., and Lottspeich, F. (1986). The acid-stable proteinase inhibitor of human mucous secretions (HUSI-I, antileukoprotease). FEBS Lett. 199, 43-48.

Sherins, R. J., and Howards, S. S. (1986). Male infertility. In Campbell's Urology., P. C. Walsh, ed. (Philadelphia: Saunders, W. B.), pp. 640-697.

Sonderegger, P., and Rathjen, F. G. (1992). REgulation of axonal growth in the vertebrate nervous system by interactions between glycoproteins belonging to two subgroups of the immunoglobulin superfamily. J. Cell Biol. 119, 1387-1394.

Soussi-Y (1996). J. Cell. Sci.

Stetler, G., Brewer, M. T., and Thompson, R. C. (1986). Isolation and sequence of a human gene encoding a potent inhibitor of leucocyte proteases. Nucl. Acids Res. 14, 7883-7896.

Whitten, W. K. (1956). Modification of the oestrous cycle of the mouse by external stimuli associated with the male. J. Endoer. 13, 399, 404.

Wiedow, O., Schroder, J. M., Gregory, H., young, J. A., and Christophers, E. (1990). Elafin: an elastase-specific inhibbitor of human skin. Purification, characterization, and complete amino acid sequence. j. Biol. Chem. 265, 14791-14795.

Wray, S., Grant, P., and Gainer, H. (1989). Evidence that cells expressing luteinizing hormone-releasing hormone mRNA in the mouse are derived from progenitor cells in the olfactory placode. proc. Natl. Acad. Sci. USA 86, 8132-8136.

Zuellig, R. A., Rader, C., Schroeder, A., Kalousek, M. B., Von Bohlen and Halbach, F., osterwalder, T., Inan, C., Stoeckli, E. T.,, Affolter, H. U., Fritz, A., and et al. (1992). The axonally secreted cell adhesion molecular axonin-1. Primary structure, immunoglobulin-like and fibronectin-type-III-like domains and glycosylphosphatidylinositol anchorage. Eur. J. Biochem. 204, 453-463.

1. A therapeutic composition comprising a pharmaceutically active amount of purified KAL protein or a biologically active part of the KAL protein.

2. The therapeutic composition according to claim 1, wherein the purified biologically active part of the KAL protein comprises at least one amino acid sequence selected among the following sequences:
the sequence beginning at the amino acid in position 182 and ending at the amino acid in position 286 of the entire amino acid sequence of the human KAL protein;
the sequence beginning at the amino acid in position 287 and ending at the amino acid in position 403 of the entire amino acid sequence of the human KAL protein;
the sequence beginning at the amino acid in position 404 and ending at the amino acid in position 541 of the entire amino acid sequence of the human KAL protein;
the sequence beginning at the amino acid in position 542 and ending at amino acid in position 662 of the entire amino acid sequence of the human KAL protein;
the sequence NH₂-RPSWYQFRVAAVNVHGTRGFTAPSKHFRSSK-COOH (32R1).

3. The therapeutic composition according to claim 1, wherein the purified KAL protein is glycosylated.

4. The therapeutic composition according to claim 1 wherein the purified KAL protein has been obtained by recombinant DNA technique.

5. The therapeutic composition according to claim 1 wherein the purified KAL protein is obtained from a culture of a CHO cell line that has been transfected by a vector carrying a nucleotide sequence coding for the KAL protein.

6. The therapeutic composition according to claim 5 wherein the transfected CHO cell line is the clone CHKAL2-3/d11 that has been deposited at CNCM under the accession number I-1792.

7. The therapeutic composition of claim 1 which is in the form of a liquid solution.

8. The therapeutic composition of claim 1 which is in the form of a gel.

9. The therapeutic composition of claim 1 which is in the form of a dry powder.

10. A CHO cell line transfected with a plasmid containing the entire 2,040 bp coding region of human KAL cDNA, as well as 56 bp and 293 bp of 5′ and 3′ non coding regions, which transfected CHO cell line is clone CHKAL2-3/d11 and has been deposited at the CNCM under the accession number I-1792.

11. The use of a therapeutic composition of claim 1 for the manufacture of a medicament for treating neuronal is disorders in a human patient selected from the group consisting of traumatic, infectious, metabolic and inherited nerve injury.

12. The use of a therapeutic composition of claim 1 for the manufacture of a medicament for treating renal disorders in a human patient selected from the group consisting of traumatic, infectious, metabolic and inherited renal injury.

13. The use of a therapeutic composition of claim 1 for the manufacture of a medicament for treating a human patient subjected to a renal transplantation.

14. The use of a therapeutic composition of claim 1 for the manufacture of a medicament for treating retinal disorders such as retinal degeneration or detachment in a human patient.

15. The use of a therapeutic composition of claim 1 for the manufacture of a medicament for treating a human patient subjected to retinal transplantation.

16. The use according to claim 13 wherein the therapeutic composition is administered by a local route.

17. A hybridoma cell line producing monoclonal antibodies directed to the recombinant human KAL protein, which hybridoma is clone 1-4 and has been deposited at CNCM under the accession number I-1791.

18. A method for detecting KAL protein in a biological sample comprising the steps of:
a) bringing into contact a biological sample and a monoclonal antibody produced by a hybridoma according to claim 17;
b) detecting the antibody-antigen complexes formed after said contact.

19. A therapeutic composition containing a pharmaceutically effective amount of a polynucleotide coding for the purified KAL protein, or coding for a protein having at least 80% homology in aminoacid sequence with the KAL protein, or coding for a protein having at least 80% homology in aminoacid sequence with a purified biologically active part of the KAL protein or coding for a protein which is recognized by antibodies directed against the purified KAL protein.

20. A therapeutic composition according to claim 19 wherein the polynucleotide encoded protein is recognized by the monoclonal antibodies produced by the hybridoma cell line that has been deposited at the CNCM on Dec. 5, 1996 under the accession number I-1791.

21. A method for the production of the purified recombinant KzL protein comprising the steps of:
a) Cultivating a prokaryotic or an eukaryotic cell that has been transfected with a vector carrying a DNA insert coding for the KAL protein or a purified biologically active part of the KAL protein or a protein which is recognized by antibodies directed against the purified KAL protein;
b) isolating the recombinant KAL protein from the culture preparation of the transfected prokaryotic or eukaryotic cell.

22. The method of claim 21 wherein the transfected cell is a eukaryotic cell.

23. The method of claim 22 wherein the transfected cell is a mammalian cell or a yeast cell.

24. The method of claim 21 wherein the transfected eukaryotic cell is the CHO cell line designated CHKAL2-3/d11 which has been deposited on Dec. 5, 1996 at the CNCM under the accession number I-1792.

25. The use of a therapeutic composition according to claim 19 for the manufacture of a medicament for treating neuronal or renal disorder in a vertebrate.

26. A method for screening ligands that bind directly or indirectly to the KAL protein or one of its biologically active derivatives comprising the steps of:
a) Preparing a complex between the KAL protein, or one of its biologically active derivatives, and a ligand that binds to the KAL protein by a method selected among the following:
preparing a tissue extract containing the KAL protein putatively bound to a natural ligand;
bringing into contact the purified KAL protein or its purified biologically active derivative with a solution containing a molecule to be tested as a candidate ligand binding to the KAL protein;
b) visualizing the complex formed between the KAL protein, or its biologically active derivative from the tissue extract and the natural ligand of the KAL protein or the complex formed between the purified KAL protein and the molecule to be tested.

27. A method for screening molecules that modulate the expression of the KAL protein comprising the steps of:
a) cultivating a prokaryotic or an eukaryotic cell that has been transfected with a nucleotide sequence encoding the KAL protein, placed under the control of its own promoter;
b) bringing into contact the cultivated cell with a molecule to be tested;
c) quantifying the expression of the KAL protein.

28. A method for screening ligands that bind to the KAL protein or to one of its biologically active derivative comprising the steps of:
a) Constructing a recombinant phage library containing human or chicken genomic DNA or cDNA.
b) bringing into contact the recombinant phages of step
c) with an immobilized purified KAL protein, or a biologically active derivative, in order to select the recombinant phages that specifically bind to the KAL protein;
c) optionally washing the bound recombinant phages in order to remove non specific binding;
d) optionally repeating step b) 2-4 times in order to select the recombinant phages that bind the most specifically to the KAL protein or the biologically active derivative.

29. A ligand capable of modulating the expression of the KAL protein expressed by a polynucleotide according to the invention, or capable of binding to the KAL protein or one of its biologically active derivatives.

30. A ligand capable of modulating the expression of the KAL protein expressed by a polynucleotide according to the invention, or capable of binding to the KAL protein or one of its biologically active derivatives which ligand is obtained according to one of the methods according to any one of claims 26 and 28.

31. A method for screening ligands that bind to the KAL protein or to one of its biologically active derivative comprising the steps of:
a) Constructing a recombinant vector library containing human or chicken genomic DNA or cDNA;
b) bringing into contact host cells transfected with the recombinant vectors of step a) with an immobilized purified KAL protein, or a biologically active part of the KAL protein.

32. A peptide comprising the following sequence NH₂-RPSWYQFRVAAVNVHGTRGFTAPSKHFRSSK-COOH (32R1).


SEQUENCE LISTING (8 sequences)

SEQ ID NO: 1 - protein, 679 aa, Homo sapiens
MVPGVPGAVLTLCLWLAASSGCLAAGPGAAAARRLDESLSAGSVQRAPCASRCLSLQITRISAFFQHFQNNGSLVWCQNHKQCSKCLEPCKESGDLRKHQCQSFCEPLFPKKSYECLTSCEFLKYILLVKQGDCPAPEKASGFAAACVESCEVDNECSGVKKCCSNGCGHTCQVPKTLYKGVPLKPRKELRFTELQSGQLEVKWSSKFNISIEPVIYVVQRRWNYGIHPSEDDATHWQTVAQTTDERVQLTDIRPSRWYQFRVAAVNVHGTRGFTAPSKHFRSSKDPSAPPAPANLRLANSTVNSDGSVTVTIVWDLPEEPDIPVHHYKVFWSWMVSSKSLVPTKKKRRKTTDGFQNSVILEKLQPDCDYVVELQAITYWGQTRLKSAKVSLHFTSTHATNNKEQLVKTRKGGIQTQLPFQRRRPTRPLEVGAPFYQDGQLQVKVYWKKTEDPTVNRYHVRWFPEACAHNRTTGSEASSGMTHENYIILQDLSFSCKYKVTVQPIRPKSHSKAEAVFFTTPPCSALKGKSHKPIGCLGEAGHVLSKVLAKPENLSASFIVQDVNITGHFSWKMAKANLYQPMTGFQVTWAEVTTESRQNSLPNSIISQSQILPSDHYVLTVPNLRPSTLYRLEVQVLTPGGEGPATIKTFRTPELPPSSAHRHLKHRHPHHYKPSPERY

SEQ ID NO: 2 - protein, 32 aa, Homo sapiens
RPSRWYQFRVAAVNVHGTRGFTAPSKHFRSSK

SEQ ID NO: 3 - protein, 32 aa, Gallus gallus
RASRWYQFRVAAVNVHGTRGFTAPSKHFRSSK

SEQ ID NO: 4 - protein, 32 aa, Danio rerio
RPGRWYQFRVAAVNVHGTRGYTIPSRHSDHLK

SEQ ID NO: 5 - DNA, 25 bp, Artificial Sequence
  Description of Artificial Sequenceprimer
cagccaatggtgcggcctcctgtcc

SEQ ID NO: 6 - DNA, 22 bp, Artificial Sequence
  Description of Artificial Sequenceprimer
tcccggcagacagcgactccgt

SEQ ID NO: 7 - protein, 16 aa, Artificial Sequence
  Description of Artificial Sequencepeptide
CSVPTKKKRRKTTDGF

SEQ ID NO: 8 - protein, 18 aa, Artificial Sequence
  Description of Artificial Sequencepeptide
CGSYANNNRYGRDPPTSV